(19) United States
(12) Patent Application Publication
(10) Pub. No.: US 2021/0205006 A1
(43) Pub. Date: Jul. 8, 2021

(54) Methods and Devices for Endoscopic Resection

(71) Applicant: Virender K. Sharma, Paradise Valley, AZ (US)

(72) Inventor: Virender K. Sharma, Paradise Valley, AZ (US)

(21) Appl. No.: 17/143,654

(22) Filed: Jan. 7, 2021

Related U.S. Application Data
(60) Provisional application No. 62/957,954, filed on Jan. 7, 2020.

(51) Int. Cl. A61B 18/14; A61B 17/00

(52) U.S. Cl. CPC A61B 18/1477; A61B 2018/00011; A61B 2017/00867; A61B 17/00234

(57) ABSTRACT

Devices for performing an endoscopic resection include at least two members, including a penetrating tip and optionally a stopper, and a coiling central portion connecting the at least two members. A first member is attached to one mucosal edge of a lesion and a second member is attached to another mucosal edge of the catheter. The device is released and the central portion is allowed to coil, pulling the first member and the second member together to contract and compress the lesion, reducing at least one dimension of the lesion. A deployment catheter for releasing the device onto a lesion includes an outer catheter and an inner catheter with mechanisms for delivering the resection device.

### CROSS-REFERENCE

The present application relies on, for priority, U.S. Patent Provisional Application No. 62/957,954, entitled “Methods and Devices for Endoscopic Resection” and filed on Jan. 7, 2020, which is herein incorporated by reference in its entirety.

### FIELD

The present specification is related generally to the field of endoscopy. More specifically, the present specification is related to devices and methods for performing an endoscopic resection.

### BACKGROUND

Endoscopic mucosal resection (EMR) or endoscopic full-thickness resection (EFTR) is a procedure used to remove early-stage cancer and precancerous growths from the lining of the digestive tract. Endoscopic mucosal or full-thickness resection is typically performed with a long, narrow tube equipped with a light and video camera. During EMR or EFTR of the upper digestive tract, the clinician passes an endoscope down the patient's throat and into the esophagus, stomach or upper part of the small intestine (duodenum). To reach the colon, the clinician may guide the tube up through the anus. The clinician then inserts instruments through the tube to perform the various resection procedures.

EMR or EFTR is usually performed to treat a health condition or collect samples of tissue during the procedure for diagnostic purposes. EMR or EFTR are less invasive alternatives to surgery for removing abnormal tissues from or in the lining of the digestive tract.

### SUMMARY

The following embodiments and aspects thereof are described and illustrated in conjunction with systems, tools and methods, which are meant to be exemplary and illustrative, and not limiting in scope. The present application discloses numerous embodiments.

The present specification discloses a device for performing an endoscopic resection comprising: a first member, wherein the first member comprises at least one first penetrating tip for piercing a first position on a lesion inside a patient; a second member, wherein the second member comprises at least one second penetrating tip for piercing a second position on the lesion inside the patient; and a central portion having a first end and a second end, wherein the first member is attached to the first end of the central portion and the second member is attached to the second of the central portion, wherein the central portion is configured to change shape from a first linear configuration to a second coiled configuration, wherein, upon attachment of the device to the lesion, the at least one first member is configured to grasp the first position, the at least one second member is configured to grasp the second position, and the central portion is configured to change shape from the first linear configuration to the second coiled configuration to contract and thereby compress the lesion and reduce at least one dimension of the lesion.

Optionally, the first and second members each comprise hooks.

Optionally, the device further comprises a deployment catheter configured to contain the device and maintain the central portion in the first linear configuration. Optionally, the central portion is configured to change shape to the second coiled configuration upon exiting the deployment catheter.

Optionally, the central portion comprises at least one of a spring, coil, or elastic band.

Optionally, the central portion comprises a shape memory material. Optionally, the shape memory material is Nitinol.

Optionally, the first position is positioned opposite the second position around an edge of the lesion.

Optionally, the device further comprises a third member, wherein the third member comprises at least one third penetrating tip for piercing a third position on the lesion inside the patient. Optionally, the first position, second position, and third position are equidistantly spaced around an edge of the lesion.

The present specification also discloses a method of performing an endoscopic resection comprising: providing a resection device comprising: a first member, wherein the first member comprises at least one first penetrating tip for piercing a first position on a lesion inside a patient; a second member, wherein the second member comprises at least one second penetrating tip for piercing a second position on the lesion inside the patient; a central portion having a first end and a second end, wherein the first member is attached to the first end of the central portion and the second member is attached to the second of the central portion, wherein the central portion is configured to change shape from a first linear configuration to a second coiled configuration; and a deployment catheter configured to contain the device and maintain the central portion in the first linear configuration; extending the resection device from a distal end of the deployment catheter such that the at least one first penetrating tip pierces into the first position; retracting the catheter such that the resection device is pulled across the lesion and the at least one second penetrating tip of the second member pierces into the second position; and removing the catheter and releasing the central portion across the lesion, allowing the central portion to change shape from the first linear configuration to the second coiled configuration to thereby compress the lesion and reduce at least one dimension of the lesion. Optionally, the method further comprises lifting the lesion using a sub-mucosal injection to lift the lesion off a muscularis of the patient. Optionally, the method further comprises using an electrosurgical device to remove the lesion. The electrosurgical device may be a needle/knife.

Optionally, the deployment catheter further comprises a lumen through which fluid may be passed and injected into or proximate the lesion. Optionally, the fluid is heated saline and a temperature of the heated saline is between 50° C. and 100° C.

Optionally, the method further comprises at least one stopper positioned proximate the first member or the second member, wherein at least one dimension of the at least one stopper is greater than a diameter of the first member or the second member.

Optionally, the central portion comprises at least one of a spring, coil, elastic band, or a shape memory material. The shape memory material may be Nitinol.

The present specification also discloses a method for performing an endoscopic resection, comprising: pushing an anchor out of a deployment catheter and positioning a first hook on a first edge of a lesion; pulling the anchor across the lesion and positioning a second hook onto a second edge of the lesion; releasing the anchor across the lesion, allowing the central portion or coil to retract pulling the first hook and the second hook together such that the two edges of the lesion are closer together.

Optionally, the method further comprises using a snare or a needle knife or any other suitable resection technique to lift the lesion once it is aggregated.

Optionally, the method further comprises using a submucosal injection of saline or another lifting agent to lift the lesion.

The present specification also discloses a distal attachment cap for use in performing an endoscopic resection and that can be connected to a distal end of an endoscope, comprising: two electrical cutting wires connected at the distal opening of the cap including an insulated ring, sphere, or ball wherein the insulated ring, sphere, or ball has an opening in the center to engage a catheter or electrosurgical knife/needle passed through the channel of the endoscope, wherein the catheter is able to manipulate the ring, sphere, ball to improve the contact of the cutting wires with a target tissue.

Optionally, the cutting wires are connected to an electrosurgical generator.

Optionally, the cutting wires have slack and the ring, sphere, ball can be extended by a distance ranging from 1 mm to 50 mm.

Optionally, the ring is offset from the center of the distal opening and configured to align with the channel of the endoscope.

Optionally, one cutting wire is an anode and a second cutting wire is a cathode.

Optionally, the cutting wires operate as an anode or a cathode while a catheter operates as the opposite electrode.

Optionally, the cutting wires operate as an anode or a cathode while an electrosurgical knife/needle operates as the opposite electrode.

The present specification also discloses a device with plurality of hooks and an expandable middle section or central portion. In embodiments, a first hook may be attached to one mucosal edge of a lesion and a second hook may be attached to another mucosal edge of the catheter allowing the connectors between the two hooks to contract and compress the lesion reducing at least one dimension of the lesion (or “aggregating” the lesion). In one embodiment, the device creates a pull from the periphery of a lesion towards the center of the lesion thereby cinching the lesion. The lesion may then be resected using an electrosurgical device. Optionally, the lesion may be lifted using a sub-mucosal injection to lift the lesion off the muscularis. Optionally, the electrosurgical device may be a snare. Optionally, the electrosurgical device may be a snare or a needle/knife or any electrosurgical instrument known in the field of endoscopic surgery. Optionally, the middle section or central portion is a spring, coil, or elastic band or any other type of expandable portion configured to pull the periphery of a lesion towards the center of a lesion. Optionally, at least one stopper is positioned on a hook wherein at least one dimension of the stopper is greater than a diameter of the hook wire. Optionally, the stopper is at least partially made of a magnetic or a ferromagnetic material.

The present specification also discloses a distal attachment cap that reversibly attaches to the distal end of an endoscope having one or more electrical cutting wires connected at the distal opening of the cap with an insulated ring, sphere, or ball wherein the insulated ring, sphere, or ball has an opening in the center to engage a catheter or electrosurgical knife/needle passed through the channel of an endoscope, wherein the catheter is configured to manipulate the ring, sphere, or ball to improve the contact of the cutting wires with the target tissue; and wherein the cutting wires are connected to an electrosurgical generator. Optionally, the knife/needle or catheter has a steerable tip with one or more degrees of motion. In embodiments, the cutting wires have are extendable such that the ring, sphere, or ball can be extended by a distance ranging from 1 mm to 50 mm. Optionally, the ring is offset from the center of the distal opening to align with the biopsy channel of the endoscope. Optionally, the ring, sphere, or ball is sized, shaped and aligned so as not to significantly interfere with the visualization with an endoscope. Optionally, a first cutting wire is an anode and a second cutting wire is a cathode. Optionally, the cutting wires operate as an anode or a cathode while a catheter operates as the opposite electrode. Optionally, the cutting wires operate as an anode or a cathode while an electrosurgical knife/needle operates as the opposite electrode. Optionally, a skin surface mounted grounding pad acts as the alternating electrode to the knife/needle and/or the cap. Optionally, the catheter has a lumen to inject a fluid on to the tissue. Optionally, the fluid is heated saline, wherein the temperature of the saline is between 50° C. and 100° C.

Optionally, the knife/needle or the cap delivers an electrosurgical RF energy greater than or equal to 1 KHz which is combined with a neurostimulating pulse of <1 KHz. The energy content of the electrosurgical pulse is higher than that of neurostimulating pulse.

The present specification also discloses a snare resection device with one or more integrated magnetic or ferromagnetic elements. In various embodiments, the magnetic or ferromagnetic elements are fixed to the wire of the snare resection device or freely mobile. In various embodiments, the polarity of the magnetic or ferromagnetic elements is arranged such that each magnetic/ferromagnetic element repels an adjacent magnetic/ferromagnetic element so the elements do not clump to each other while on the snare, thereby preventing the interruption of the opening and closing function of the snare by clumped elements. In various embodiments, the magnetic/ferromagnetic elements are designed to engage with magnetic or ferromagnetic elements of a retraction hook and coil (anchor) device.

The aforementioned and other embodiments of the present specification shall be described in greater depth in the drawings and detailed description provided below.

### BRIEF DESCRIPTION OF THE DRAWINGS

These and other features and advantages of the present invention will be further appreciated, as they become better understood by reference to the detailed description when considered in connection with the accompanying drawings:

FIG. 1 is an illustration of a resection device that uses anchoring by a first member and a second member, in accordance with embodiments of the present specification;

FIG. 2A is a pictorial illustration of a plurality of steps of a method for using a resection device with first and second members or retraction hooks as described in FIG. 1, in an embodiment of the present specification;

FIG. 2B is a flowchart of the plurality of steps of the method of FIG. 2A, in an embodiment of the present specification;

FIG. 3 illustrates embodiments of resection device designs as used in embodiments of the present specification;

FIG. 4 illustrates embodiments of a delivery catheter as employed in an embodiment of the present specification;

FIG. 5 illustrates a resection device loaded onto an inner catheter of a delivery device, as used in an embodiment of the present specification;

FIG. 6 illustrates two resection devices applied across a lesion in accordance with embodiments of the present specification;

FIG. 7 illustrates various resection devices as employed in embodiments of the present specification;

FIG. 8 illustrates exemplary resection device dimensions as employed in embodiments of the present specification;

FIG. 9 illustrates exemplary resection device dimensions as employed in other embodiments of the present specification;

FIG. 10A illustrates three-member or hook resection devices as employed in embodiments of the present specification;

FIG. 10B illustrates three-member or hook resection devices as employed in other embodiments of the present specification;

FIG. 10C is a flowchart of a plurality of exemplary steps of a method of deploying a three-member or hook resection device, in accordance with some embodiments of the present specification;

FIG. 11 illustrates various piercing member or hook embodiments as employed in embodiments of the present specification;

FIG. 12 illustrates various non-piercing member or hook embodiments as employed in embodiments of the present specification;

FIG. 13 illustrates an embodiment of a member or hook with an insulating covering as employed in embodiments of the present specification;

FIG. 14 illustrates an embodiment of a central portion for joining at least two members or hooks and providing elasticity for aggregating a lesion;

FIG. 15 illustrates a connection between at least two members or hooks and at least one central portion or coil, in an embodiment of the present specification;

FIG. 16 illustrates a resection device, as used in embodiments of the present specification;

FIG. 17 illustrates various embodiments of connectors that may be employed for connecting a member or hook and a central portion or coil in embodiments of the present specification;

FIG. 18 illustrates an exemplary configuration of an electrosurgical unit as employed in embodiments of the present specification;

FIG. 19A illustrates an exemplary configuration of an electrosurgical knife as employed in embodiments of the present specification;

FIG. 19B is a longitudinal cross-sectional view of a heating chamber including assembled first and second electrodes, in accordance with some embodiments of the present specification;

FIG. 20A is an illustration of a snare device as used in a resection procedure in embodiments of the present specification;

FIG. 20B is an embodiment of a magnet snare device configured to operate in conjunction with a resection device of the present specification;

FIG. 20C illustrates a resection of a lesion using a magnet snare device in conjunction with a resection device, in accordance with embodiments of the present specification;

FIG. 20D is a flowchart of a plurality of exemplary steps of a method of using the snare device of FIG. 20A with a resection device in a resection procedure, in accordance with some embodiments of the present specification;

FIG. 20E is a flowchart of a plurality of exemplary steps of a method of using the magnet snare device of FIG. 20B with a three-member or hook resection device in a resection procedure, in accordance with some embodiments of the present specification;

FIG. 20F illustrates a variable size snare device for use in conjunction with the resection devices of the present specification;

FIG. 21 is an illustration of a submucosal dissection procedure using a resection device in embodiments of the present specification;

FIG. 22 is a diagram of a directional grounding cap for use with embodiments of the present specification;

FIG. 23 is a diagram showing the function of the directional grounding cap shown in FIG. 22 for use with embodiments of the present specification;

FIG. 24 is a diagram of an embodiment of a bipolar cap dissector for use with embodiments of the present specification; and

FIG. 25 is a diagram of another embodiment of a bipolar cap dissector for use with embodiments of the present specification.

### DETAILED DESCRIPTION

The present specification is directed toward devices and methods for performing an endoscopic resection (ER) or endoscopic full-thickness resection (EFTR) of a lesion. The present specification discloses resection devices that use anchoring by a first member and a second member. The first and second members are joined by a central portion configured to coil after deployment, pulling the first and second members, which have grasped portions of tissue of a lesion in a body, together, thereby contracting and compressing the lesion, and reducing at least one dimension of the lesion. This function of aggregating or cinching the lesion makes the lesion easier to remove using a snare or electrocautery device.

The present specification is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

In the description and claims of the application, each of the words “comprise” “include” and “have”, and forms thereof, are not necessarily limited to members in a list with which the words may be associated. It should be noted herein that any feature or component described in association with a specific embodiment may be used and implemented with any other embodiment unless clearly indicated otherwise.

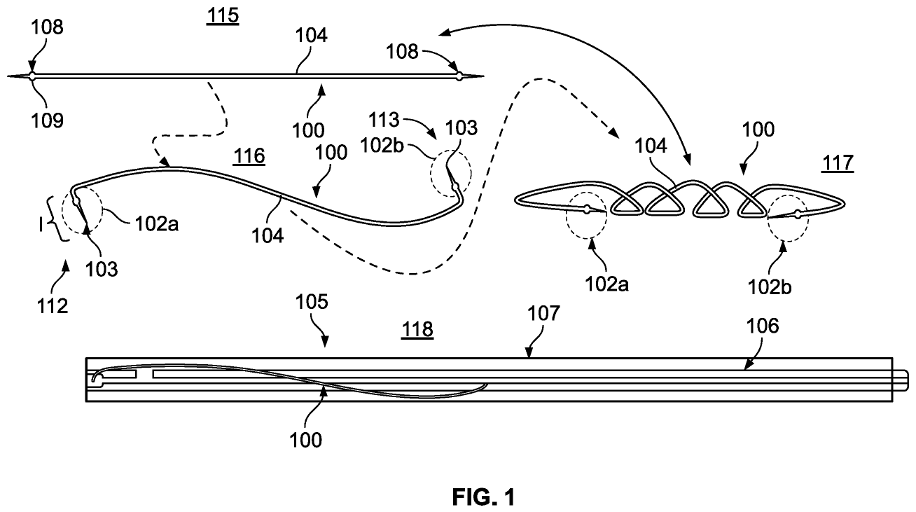

FIG. 1 is an illustration of a resection device 100 that uses anchoring by a first member 102a and a second member 102b, in accordance with embodiments of the present specification. In embodiments, the first member 102a and second member 102b comprise retraction hooks. In embodiments, the first and second members or retraction hooks 102a, 102b may be deployed using a catheter structure 105. In embodiments, the catheter 105 includes an inner catheter 106 which is configured to hold and deploy the resection device 100 and an outer catheter 107 which coaxially sheaths the inner catheter 106 and is used to deliver the inner catheter 106 and resection device 100 to a target tissue. The outer catheter 107 includes an atraumatic distal end and outer surface and is retracted once positioned proximate the target to tissue to expose the inner catheter 106 and resection device 100.

In embodiments, the first and second member or each retraction hook 102a, 102b includes a penetrating tip 103 for piercing body tissues. In embodiments, a first member or retraction hook 102a is positioned on a first or distal end 112 of a central portion 104 (such as a coil) of the device 100 and a second member or retraction hook 112b is positioned on a second or proximal end 113 of the central portion. The members or retraction hooks 102a, 102b are configured to be used as anchors when the central portion 104 coils, thereby drawing two pierced ends of tissue together. In embodiments, the first and second members or retraction hooks 102a, 102b further include a stopper 108 between the penetrating tip 103 and the central portion 104 to provide a stop point for piercing tissue. Optionally, at least one stopper 108 is positioned on each hook 102a, 102b wherein at least one dimension of the stopper 108 is greater than a diameter of the hook wire 109. In some embodiments, the stopper 108 has a diameter in a range of 0.5 to 5 mm. In some embodiments, a length/of a retraction hook 102a, 102b, from an end of a stopper 108 adjacent the central portion 104 to an end of a penetrating tip 103 opposite said stopper end, is in a range of 1 to 10 mm. In some embodiments, the stoppers 108 are composed of a ferromagnetic or magnetic element. The magnetic element may be a rare earth magnet. In embodiments, the stopper 108 is covered with an insulative layer such as a ceramic layer or PTFE. The resection device 100 is loaded on to the inner catheter 106 with mechanisms for deploying and positioning the two retraction hooks 102a, 102b into one or more targeted areas in an organ.

A first view 115 shows the resection device 100 in a first, fully extended, linear configuration. The resection device 100 transitions from the first, fully extended, linear configuration shown in view 115 to a second, fully coiled configuration shown in view 117 after the second member or retraction hook 102b grasps or engages a second portion of a body tissue, such as a lesion, and the central portion 104 fully coils. View 116 shows the resection device 100 during its transformation, in a partially coiled or curved shape, as the first member or retraction hook 102a grasps or engages a first portion of a body tissue or lesion and the coil is being deployed from the inner catheter. In embodiments, the resection device 100 undergoes a 10% to 90% reduction in length as it transitions from the first, fully extended linear configuration shown in view 115 to the second, fully coiled configuration shown in view 117. View 118 shows the resection device 100 loaded on an inner catheter 106 of a catheter structure 105. In embodiments, the resection device 100, particularly the central portion 104, has shape memory properties to facilitate the change in configurations. In some embodiments, the resection device 100, or only the central portion 104, is composed of Nitinol. In some embodiments, the resection device 100, or only the central portion 104, is composed of a temperature sensitive material which changes shape when exposed to certain temperatures, for example, the normal temperature of a human body. In some embodiments, the central portion 104 is composed of an elastic material such as silicone, latex rubber, or latex free nitrile.

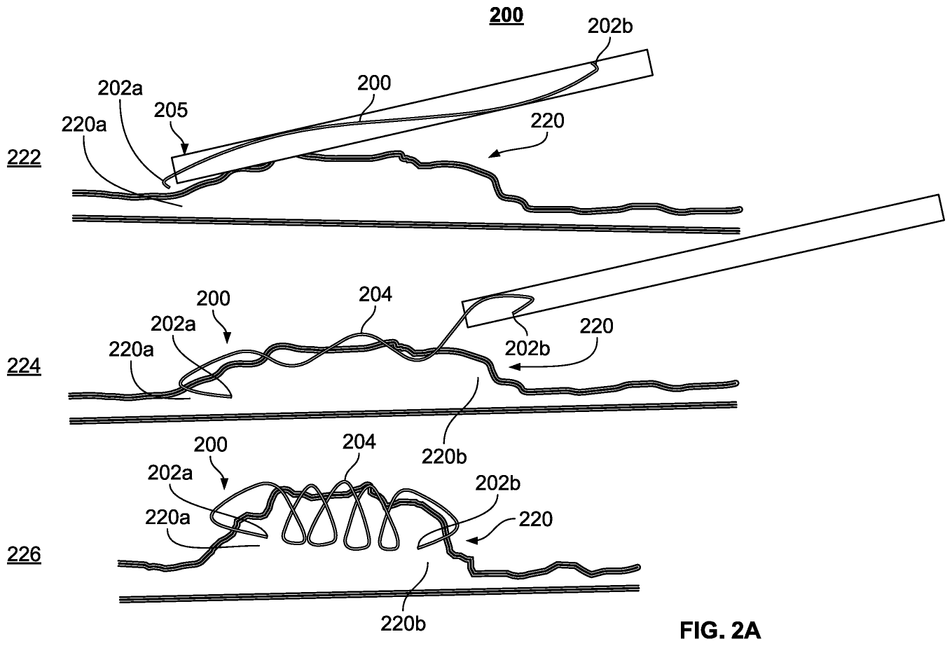

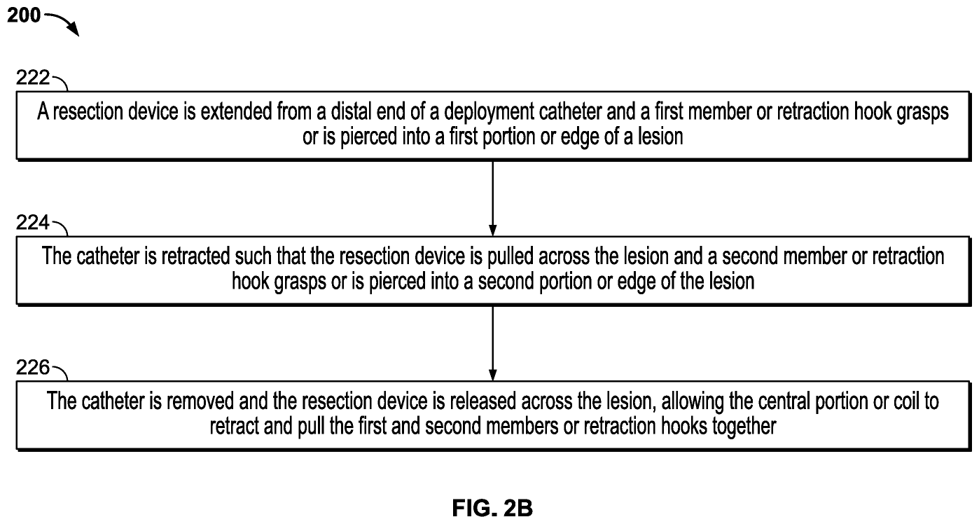

FIG. 2A is a pictorial illustration of a plurality of steps of a method 200 for using a resection device with first and second members or retraction hooks as described in FIG. 1, while FIG. 2B is a flowchart of the plurality of steps of the method 200, in an embodiment of the present specification. Referring now to FIGS. 2A and 2B, at step 222, a resection device 200 (including the first and second members or retraction hooks 102a, 102b and central portion 104 of FIG. 1) is extended from a distal end of a deployment catheter 205 and a first member or retraction hook 202a is pierced into or grasps a first portion or edge 220a of a lesion 220 at a first position. At step 224, the catheter 205 is retracted such that the resection device 200 is pulled across the lesion 220 and a second member or retraction hook 202b is pierced into or grasps a second portion or edge 220b of the lesion 220 at a second position. In embodiments, the second position is on an opposite edge of the lesion 220 relative to the first position. At step 226, the catheter 205 is removed and the resection device 200 is released across the lesion 220, allowing the central portion or coil 204 to retract and pull the first member or retraction hook 202a and the second member or retraction hook 202b together such that the two edges 220a, 220b of the lesion 220 are closer together and the edges 220a, 220b of the lesion 220 are pulled toward a center of the lesion 220, reducing at least one dimension of the lesion 220.

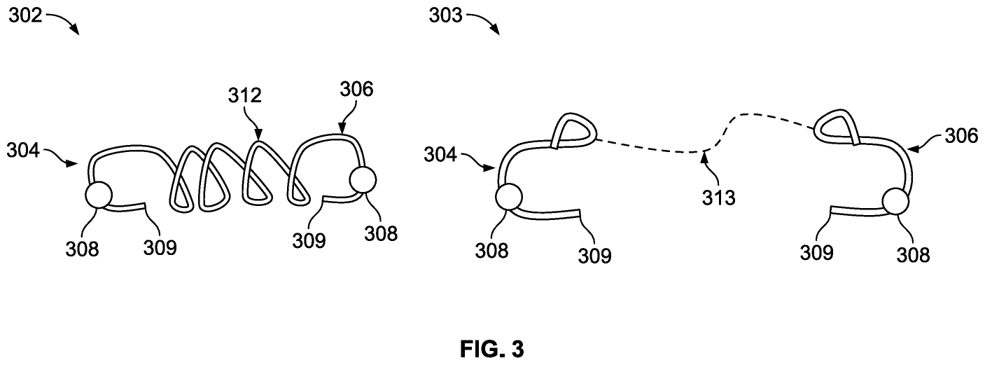

FIG. 3 illustrates embodiments of resection device designs as used in embodiments of the present specification. In embodiments, a resection device 302 comprises a first member or retraction hook 304 positioned at a first end of the resection device 302 and a second member or retraction hook 306 positioned at a second end of the resection device 302 opposite the first end. The first member or retraction hook 304 comprises a first penetrating tip 307 and a first stopper 308, and the second member or retraction hook 306 comprises a second penetrating tip 309 and a second stopper 310. The two members or hooks 304, 306 are connected by a central portion 312 which comprises a coil fabricated from a material having a shape memory, such as Nitinol. In another embodiment, a resection device 303 comprises a first member or retraction hook 304 position at a first end of the resection device 302 and a second member or retraction hook 306 positioned at a second end of the resection device 302 opposite the first end. The first member or retraction hook 304 comprises a first penetrating tip 307 and a first stopper 308, and the second member or retraction hook 306 comprises a second penetrating tip 309 and a second stopper 310. The two members or hooks 304, 306 are connected by a central portion 313 which comprises an elastic connector made of silicone, latex rubber or latex free nitrile. In embodiments, the stoppers 308 are fabricated from ceramic, PTFE, silicone, glass, SST, nitinol or a ferromagnetic or magnetic material such as a rare earth magnet. In embodiments, the stoppers have a diameter ranging from 0.5 to 5 mm.

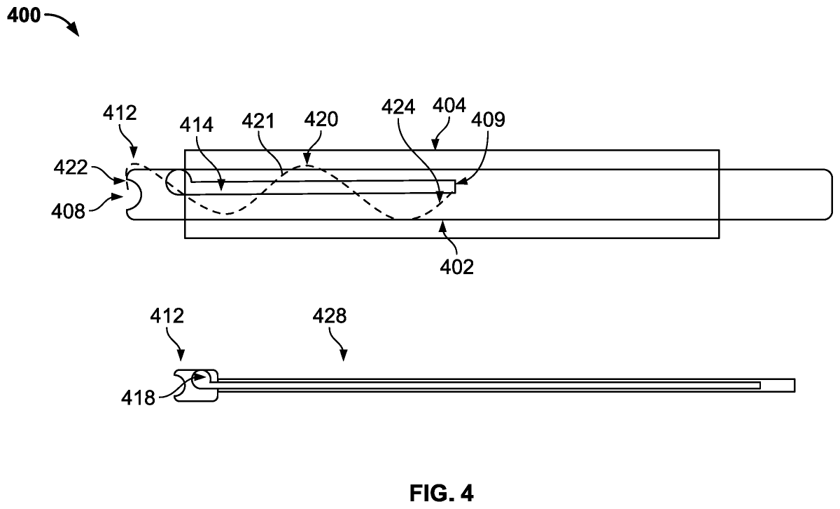

FIG. 4 illustrates embodiments of a delivery catheter as employed in an embodiment of the present specification. In embodiments, the delivery catheter 400 comprises an inner catheter 402 and an outer catheter 404. In embodiments, a distal tip 412 of the inner catheter 402 includes at least one first groove 408 configured to engage with a first member or retraction hook 422 of a resection device 420 to secure a first end of the resection device 420. The inner catheter 402 further includes a slit 414 extending proximally from the at least one first groove 408 configured to slidably receive the first member retraction hook 422, a second member or second retraction hook 424, and/or a portion of the central portion 421 of the resection device 420, and at least one second groove 409 positioned at the end of the slit 414 configured to engage with the second member or retraction hook 424 of the resection device 420 to secure a second end of the resection device 420 opposite the first end. In one embodiment, shown in inner catheter 402, the at least one first groove 408 is positioned at the end of the distal tip 412 of the inner catheter 402. In another embodiment, shown in inner catheter 428, the at least one first groove 418 is positioned on a side of the distal tip 412 of the inner catheter 428.

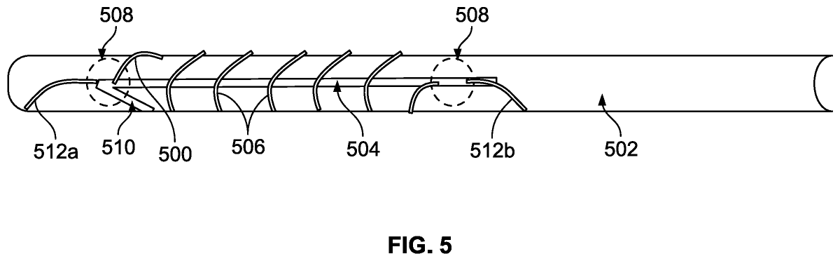

FIG. 5 illustrates a resection device 500 loaded on another embodiment of an inner catheter 502 of a catheter delivery device. In an embodiment, the resection device 500 is pre-loaded onto an inner catheter 502. In an embodiment, a rounded contour of the inner catheter 502 houses the stoppers 508 of the resection device 500 while the central portion 506 loops around the inner catheter 502. A slit 504 is provided along the length of the inner catheter 502 to slidably receive and house at least a portion of the central portion 506. The slit 504 allows the central portion 506 to slide along the inner catheter 502, such that it can be pushed out of an outer catheter (shown as 404 in FIG. 4). The slit 504 has a hook configuration 510 at one end to prevent inadvertent slippage of the central portion 506. The hook configuration 510 of the slit 504 also allows for hooking and pulling the first and second members or retraction hooks 512a, 512b on the ends of the central portion 506 of the resection device.

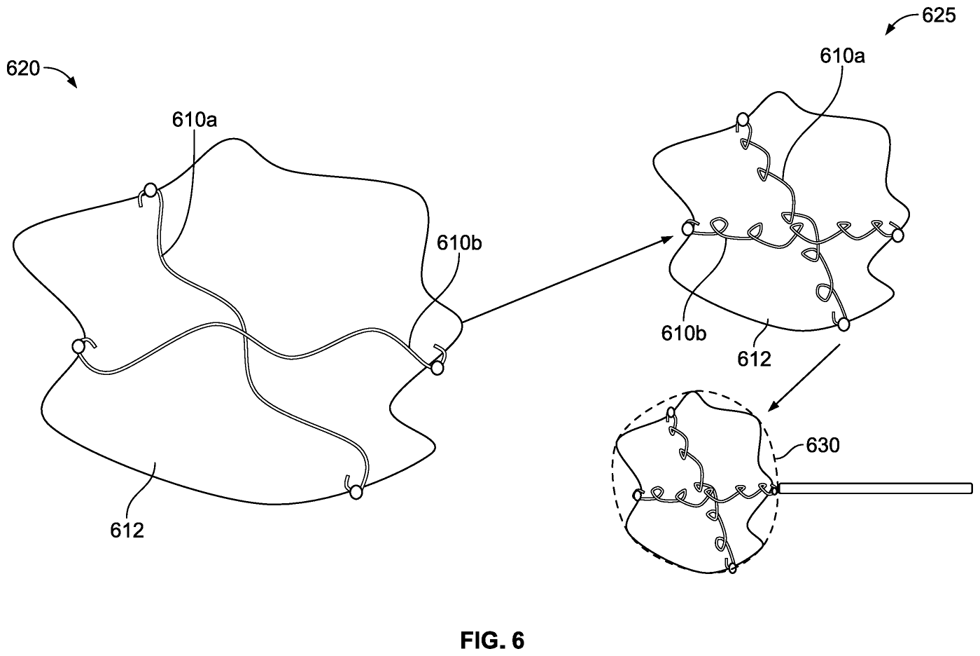

FIG. 6 illustrates a resection using two resection devices 610a, 610b (each including the first and second members or retraction hooks 102a, 102b and central portion 104 of FIG. 1) applied across a lesion 612 in a pre-deployment configuration 620 and a post-deployment configuration 625 as used in an embodiment of the present specification, also as described with respect to FIG. 2. Once the lesion 612 is “aggregated”, or its dimensions are reduced, the lesion 612 is then resected using a snare 630 or a needle knife or any other suitable resection technique. In addition, in embodiments, the lesion 612 can be lifted using a submucosal injection of saline or another lifting agent. In the case of endoscopic dissection, the resected edge of the lesion 612 is pulled toward the center of the lesion 612, lifting the resected edge to move away from the point of resection toward the center of the lesion 612, thereby exposing the plane of dissection for easy access for continued dissection.

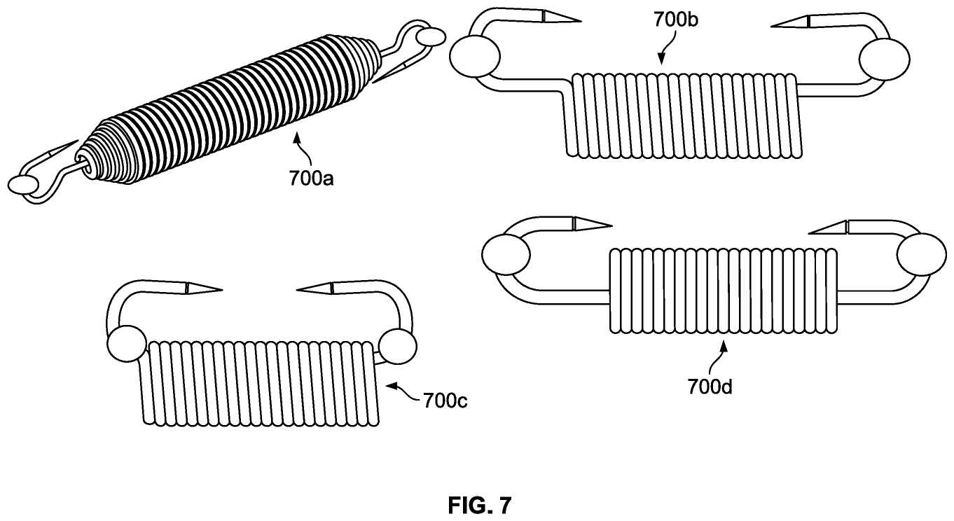

FIG. 7 illustrates first, second, third and fourth configurations 700a, 700b, 700c, 700d of resection devices as employed in embodiments of the present specification.

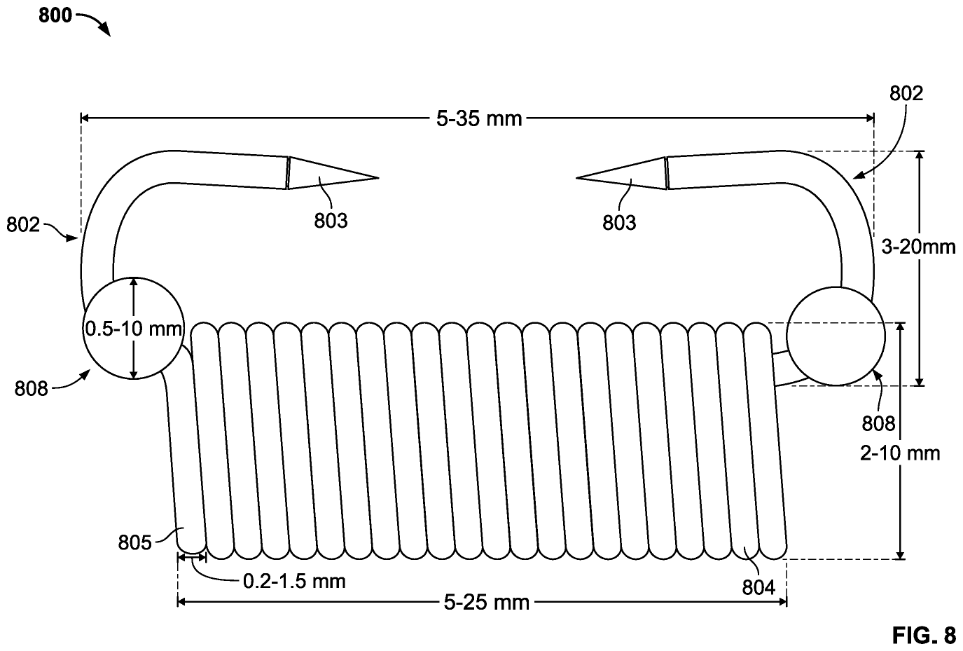

FIG. 8 illustrates exemplary dimensions of a resection device 800 as employed in various embodiments of the present specification. In embodiments, the resection device 800 has a total length ranging from 5 to 35 mm, and a length of the central portion 804, in the deployed, fully coiled configuration, ranging from 5 to 25 mm. In embodiments, the central portion 804, when in the deployed, fully coiled configuration, has a width ranging from 2 to 10 mm. In embodiments, a single loop of coil 805, when the resection device 800 is in the deployed, fully coiled configuration, has a length ranging from 0.2 to 1.5 mm. In embodiments, each stopper 808 has a diameter in a range of 0.5 to 10 mm. In embodiments, each member or retraction hook 802, including the stopper and penetrating tip 803, has a length, measure extending away from the central portion 804, ranging from 3 to 20 mm.

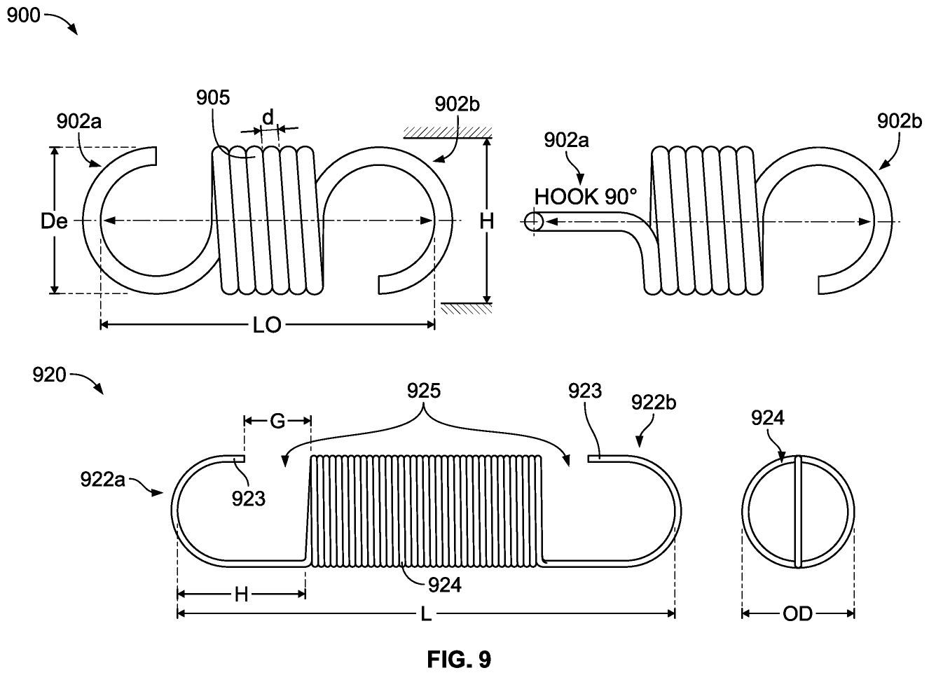

FIG. 9 illustrates exemplary dimensions of resection devices 900, 920 as employed in other embodiments of the present specification. In embodiments, resection device 900 has an overall width De ranging from 3 to 20 mm when in the deployed, fully coiled configuration and is configured to fit within a space having a height H in a range of 3 to 25 mm. The resection device 900 has a length LO from an inner surface of a first member or retraction hook 902a to an inner surface of a second member or retraction hook 902b ranging from 5 to 35 mm. In embodiments, a single loop of coil 905, when the resection device 900 is in the deployed, fully coiled configuration, has a length d ranging from 0.2 to 1.5 mm. In embodiments, the first member or retraction hook 902a is positioned in a first plane offset from a second plane, wherein the second member or retraction hook 902b is positioned, by 90 degrees. In embodiments, resection device 920 has an overall length L, when in a deployed, fully coiled configuration, ranging from 5 to 35 mm. The first member or retraction hook 922a and second member or retraction hook 922b each have a height H, extending from an end of the central portion 924 to a curved end of each hook 922a, 922b, ranging from 1.5 to 15 mm, when the resection device is in the deployed, fully coiled configuration. A gap 925 is present between each end of the central portion and each penetrating tip 923 of each hook, and has a length G ranging from 1 to 10 mm, when the resection device 920 is in the deployed, fully coiled configuration. The central portion 924 forms a coil having an outside diameter OD ranging from 3 to 35 mm when the resection device 920 is in the deployed, fully coiled configuration.

As discussed earlier with reference to FIG. 1, the resection devices of the present specification, in embodiments, include members having penetrating or sharp tips for grasping or piercing an organ or, in other embodiments, have members including blunt tips to engage the organ surface or a defect in the organ surface. In all embodiments, the penetrating tips or blunt tips anchor the members to the organ surface. In some embodiments, the blunt tips may be placed into a defect created in the lining of the organ using a separate device or electrocautery. The resection device has stopping mechanisms or stoppers on each hook which prevent the member or hooks from burying too deep into the organ while the members or hooks are pulled towards each other. The pulling action causes a cinching force rather than burying the hooks deep, avoiding perforation of an organ wall.

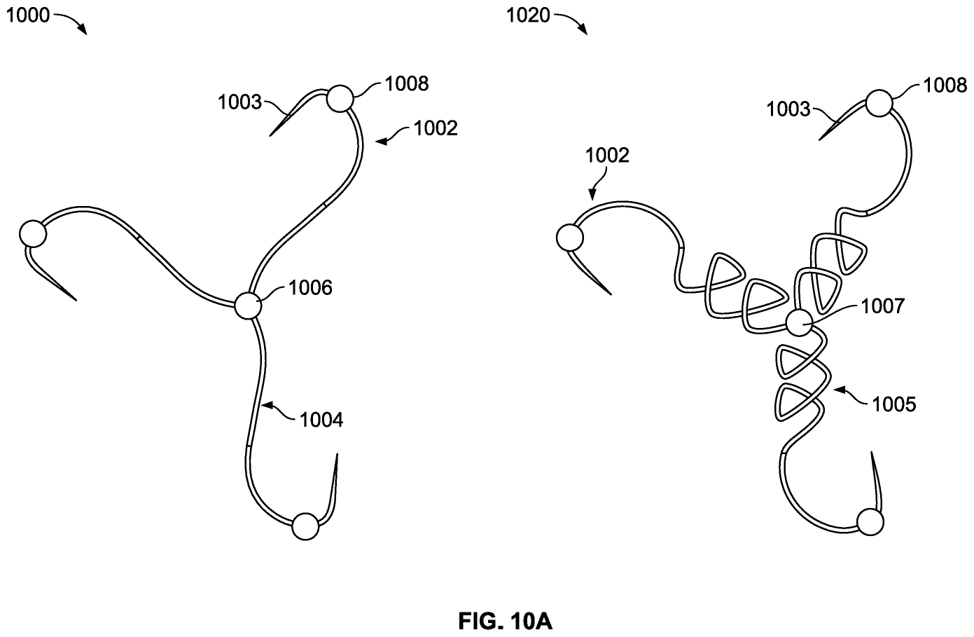

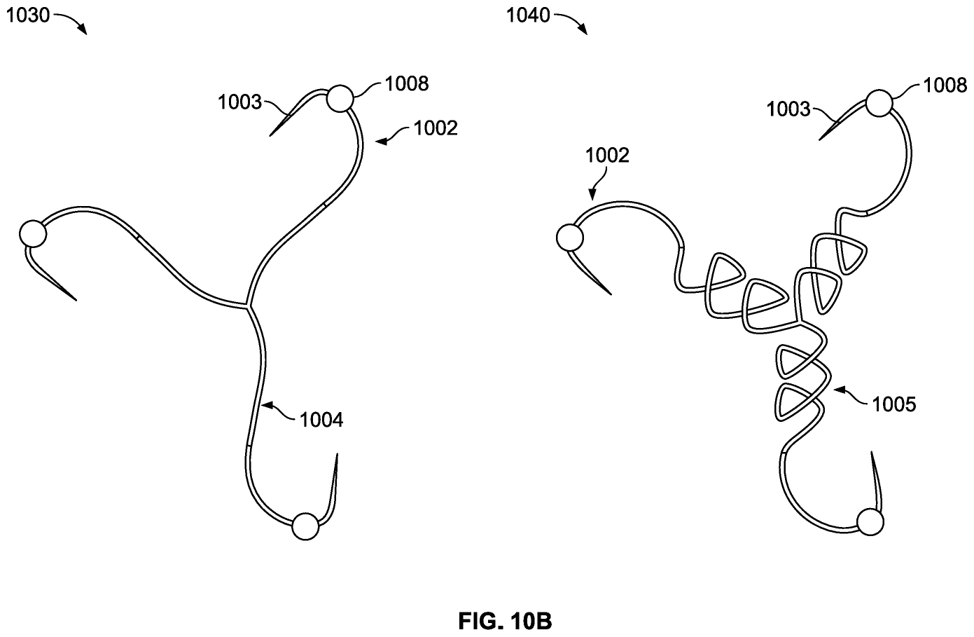

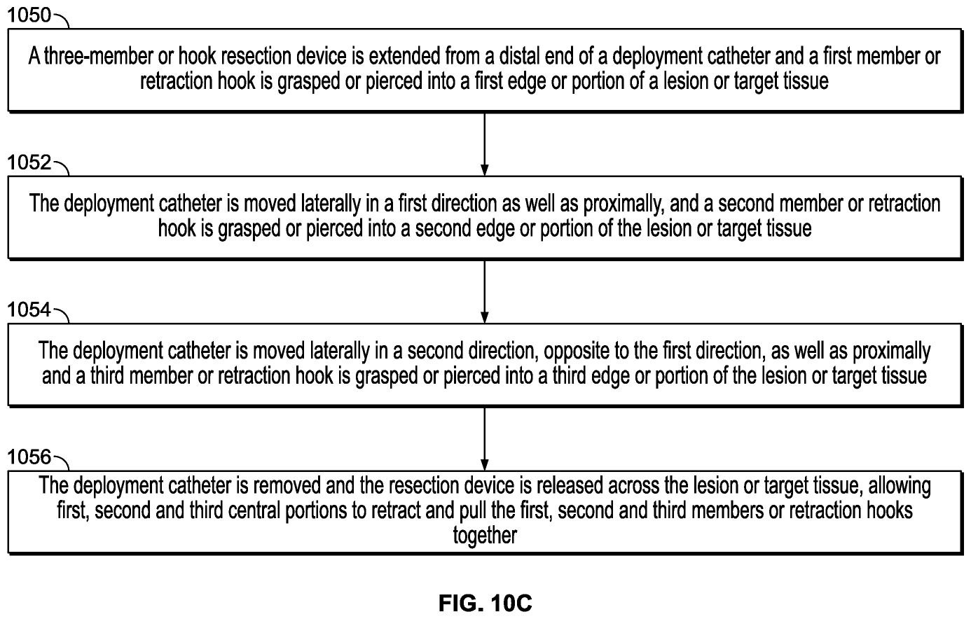

FIG. 10A illustrates three-member or hook resection devices 1000, 1020 as employed in embodiments of the present specification. Anchor 1000 includes three members or retraction hooks 1002, each comprising at least one stopper 1008 and a penetrating tip 1003 for penetrating portions of a target tissue or lesion at first, second, and third positions. In embodiments, the first position, second position, and third position are equidistantly spaced around an edge of the lesion. Each member or retraction hook 1002 is connected, via a central portion 1004, to a central connection hub 1006. In embodiments, each central portion 1004 comprises an elastic connector. In other embodiments, referring to FIG. 10B and three-member or hook resection devices 1030, 1040, the central connection hub is not included and the ends of each central portion 1004, opposite each member or hook 1002, are attached together, for example, via suturing, tying, gluing, or welding. Referring again to FIG. 10A, resection device 1020 includes three member or retraction hooks 1002, each comprising at least one stopper 1008 and a penetrating tip 1003. Each member retraction hook 1002 is connected, via a central portion 1005, to a central connection hub 1007. In embodiments, each central portion 1005 comprises a Nitinol connector. In embodiments, central connection hub 1006, 1007 comprises a ball and socket joint to allow the elastic connector central portions 1004 and hooks 1002 of resection device 1000, or the Nitinol connector central portion 1005 and hooks 1002 of resection device 1020, to articulate relative to the central connection hub 1006, 1007. The hooks 1002 are configured to reversibly engage with an inner member of a delivery catheter which is used for placement and repositioning of the hooks 1002 as needed.

FIG. 10C is a flowchart of a plurality of exemplary steps of a method of deploying a three-member or hook resection device (such as the device 1000, 1020, 1030 or 1040), in accordance with some embodiments of the present specification. At step 1050, the three-member or hook resection device is extended from a distal end of a deployment catheter and a first member or retraction hook is grasped or pierced into a first edge or portion of a lesion or target tissue. At step 1052, the deployment catheter is moved laterally in a first direction as well as proximally, and a second member or retraction hook is grasped or pierced into a second edge or portion of the lesion or target tissue. At step 1054, the deployment catheter is moved laterally in a second direction, opposite to the first direction, as well as proximally and a third member or retraction hook is pierced into a third edge or portion of the lesion or target tissue.

At step 1056, the deployment catheter is removed and the resection device is released across the lesion or target tissue, allowing first, second and third central portions (each of which connects the first, second and third members or retraction hooks to a central connection hub or are attached together) to retract and pull the first, second and third members or retraction hooks together such that the first, second and third edges or portions of the lesion or target tissue are closer together and the edges or portions of the lesion or target tissue are pulled approximately toward a center of the lesion or target tissue.

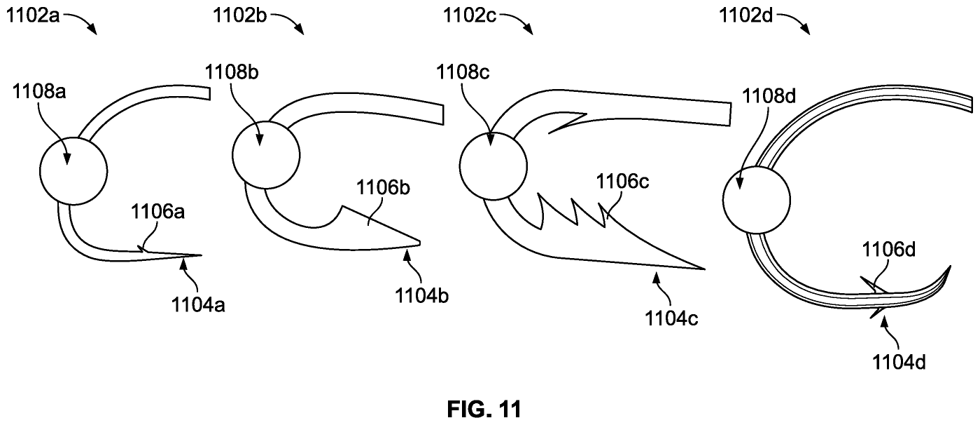

FIG. 11 illustrates various implementations of penetrating or piercing tips 1104a, 1104b, 1104c, 1104d of members or retraction hooks 1102a, 1102b, 1102c, 1102d of resection devices, in accordance with some embodiments of the present specification. Each penetrating tip includes at least one first sharp portion extending in a first direction and at least one second sharp portion extending in an opposing direction to create a barb 1106a, 1106b, 1106c, 1106d configured to pierce a body tissue. Each retraction hook 1102a, 1102b, 1102c, 1102d further comprises at least one stopper 1108a, 1108b, 1108c, 1108d.

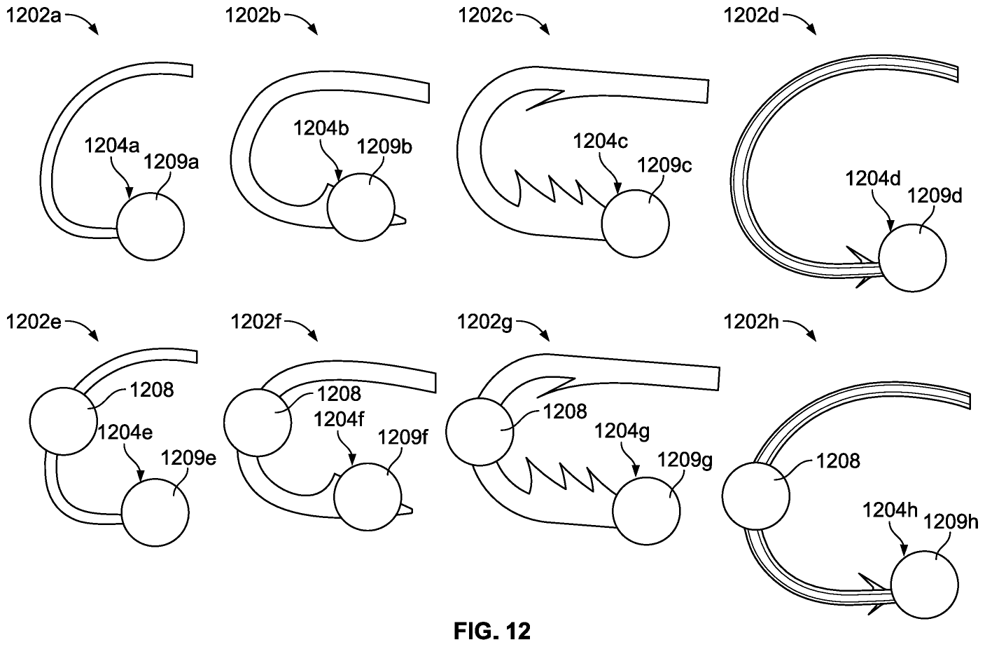

FIG. 12 illustrates various implementations of non-penetrating or non-piercing tips 1204a, 1204b, 1204c, 1204d, 1204e, 1204f, 1204g, 1204h of members or retraction hooks 1202a, 1202b, 1202c, 1202d, 1202e, 1202f, 1202g, 1202h as employed in embodiments of the present specification. The non-penetrating or non-piercing tips 1204a, 1204b, 1204c, 1204d, 1204e, 1204f, 1204g, 1204h include blunt ends 1209a, 1209b, 1209c, 1209d, 1209e, 1209f, 1209g, 1209h configured to engage an organ through a separate incision or defect created by a different tool such as an electrosurgical knife or cautery tip. The non-penetrating or non-piercing tips 1204a, 1204b, 1204c, 1204d, 1204e, 1204f, 1204g, 1204h are configured to hold the hooks in place in defects or walls in the lining of the tissue and do not penetrate the tissue. Referring to FIG. 12, members or retraction hooks 1202e, 1202f, 1202g, 1202h each include at least one stopper 1208. In the some embodiments, a stopper material or element is used to create the blunt end 1209a, 1209b, 1209c, 1209d, 1209e, 1209f, 1209g, 1209h.

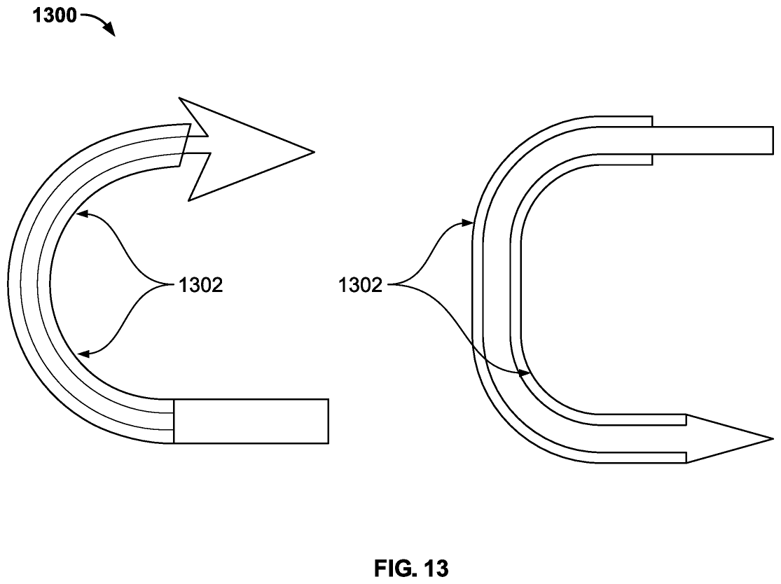

FIG. 13 illustrates an embodiment of a member or retraction hook 1300 of a resection device, with an insulating covering 1302 as employed in embodiments of the present specification. This embodiment prevents electricity from an electrosurgical instrument in contact with the resection device to transmit the electricity into the tissue causing inadvertent tissue injury in and around the resection device.

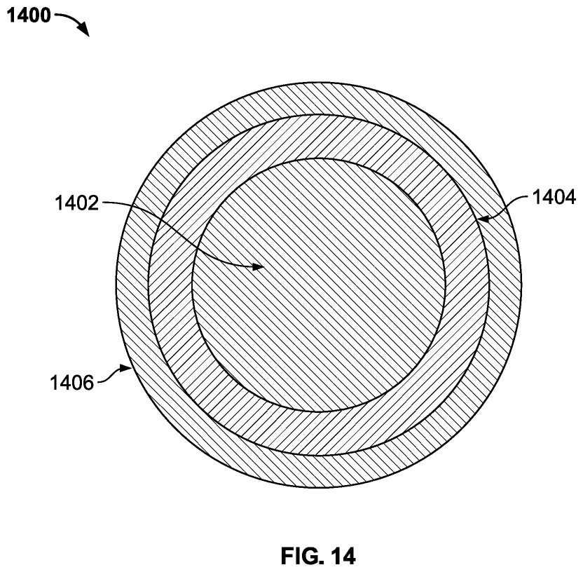

FIG. 14 illustrates a front to back cross-sectional view of an embodiment of a central portion 1400 for joining at least two members or retraction hooks of a resection device and providing elasticity for aggregating a lesion. In embodiments, the central portion 1400 comprises a core 1402, preferably fabricated from an elastic material such as silicone, Teflon, rubber or latex. A metal or alloy coil 1404 surrounds the core 1402. Finally, an elastic sleeve 1406 is provided to surround the elastic core/coil tube 1402, 1404. Preferably, the sleeve 1406 is fabricated from an insulating material to insulate the coil 1400 from an electrosurgical instrument.

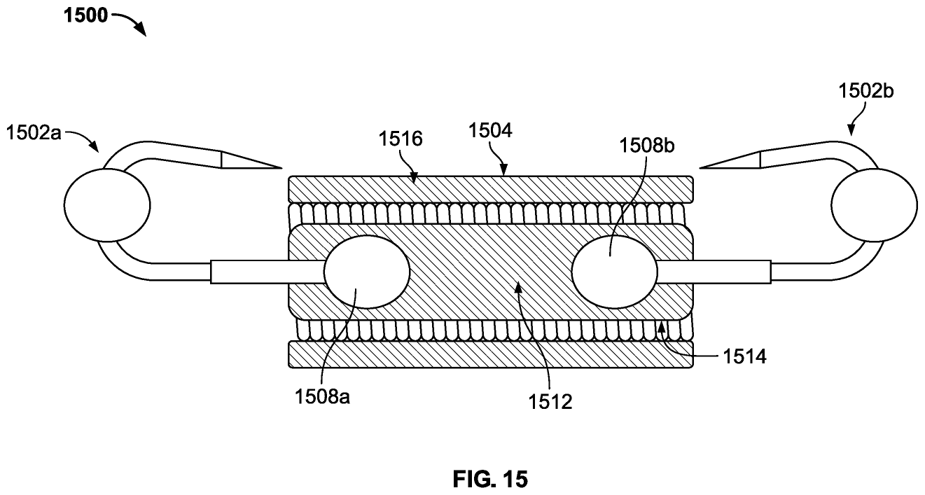

FIG. 15 illustrates side cross-sectional view of a connection between two members or retraction hooks 1502a, 1502b and a central portion 1504 of a resection device 1500, in accordance with some embodiments of the present specification. In embodiments, a central portion 1504 comprises a core 1512, preferably fabricated from an elastic material. A metal or alloy coil 1514 surrounds the core 1512. Finally, an elastic sleeve 1516 is provided to surround the elastic core/coil tube 1512, 1514. Preferably, the sleeve 1516 is fabricated from an insulating material. In embodiments, ball and socket joints 1508a, 1508b are provided at each end of the central portion 1504 to attach the members or retraction hooks 1502a, 1502b and allow for the members or retraction hooks 1502a, 1502b to articulate relative to the central portion 1504. In addition, the ball and socket joints 1508a, 1508b are composed of, or covered by, an insulating material to electrically insulate the members or hooks 1502a, 1502b from the central portion 1504.

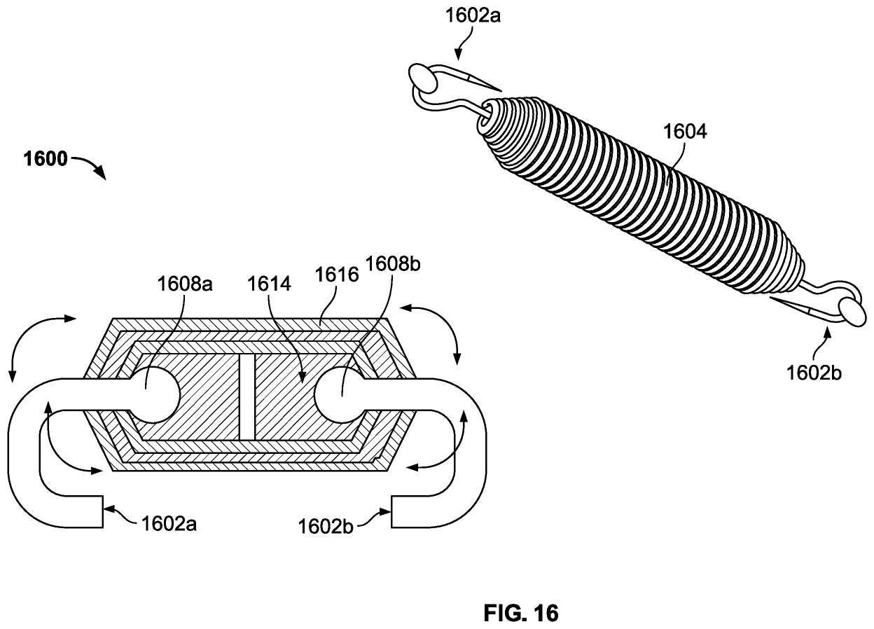

FIG. 16 illustrates side cross-sectional view of a resection device 1600, according to some embodiments of the present specification. The resection device 1600 includes a central portion 1604 comprising a coil 1614 coated with an elastic material 1616. In an embodiment, ball and socket joints 1608a, 1608b allow for articulation of the members or retraction hooks 1602a, 1602b with respect to the central portion 1604 in one or more directions and in a range of 10 degrees to 90 degrees. Advantageously, a long axis of the hooks 1602a, 1602b can rotate relatively freely relative to a long axis of the central portion 1604, helping in anchoring in different planes or directions in an irregular surface of a lesion or an organ.

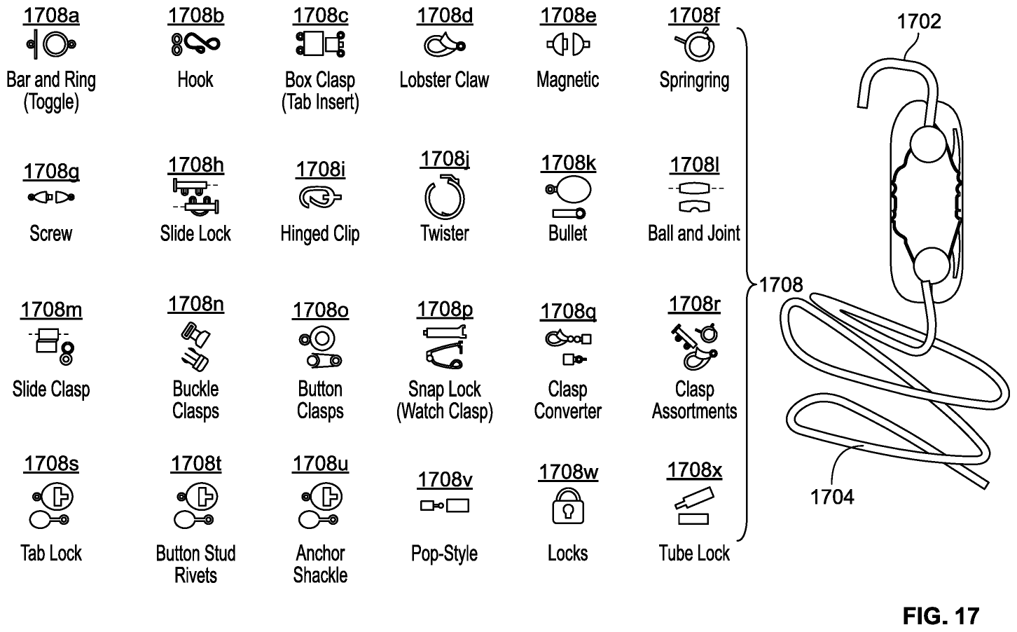

FIG. 17 illustrates various embodiments of connectors 1708 that may be employed for connecting a member or hook 1702 to a central portion or coil 1704 of a resection device, in accordance with embodiments of the present specification. In various embodiments, connectors 1708 include, but are not limited to, bar and ring (toggle) 1708a, hook 1708b, box clasp 1708c, lobster claw 1708c, magnetic 1708e, springring 1708f, screw 1708g, slide lock 1708h, hinged clip 1708i, twister 1708j, bullet 1708k, ball and joint 17081, slide clasp 1708m, buckle clasps 1708n, button clasps 1708o, snap lock (watch clasp) 1708p, clasp converter 1708q, clasp assortments 1708r, tab lock 1708s, button stud rivets 1708t, anchor shackle 1708u, pop-style 1708v, locks 1708w, and tube lock 1708x type connectors.

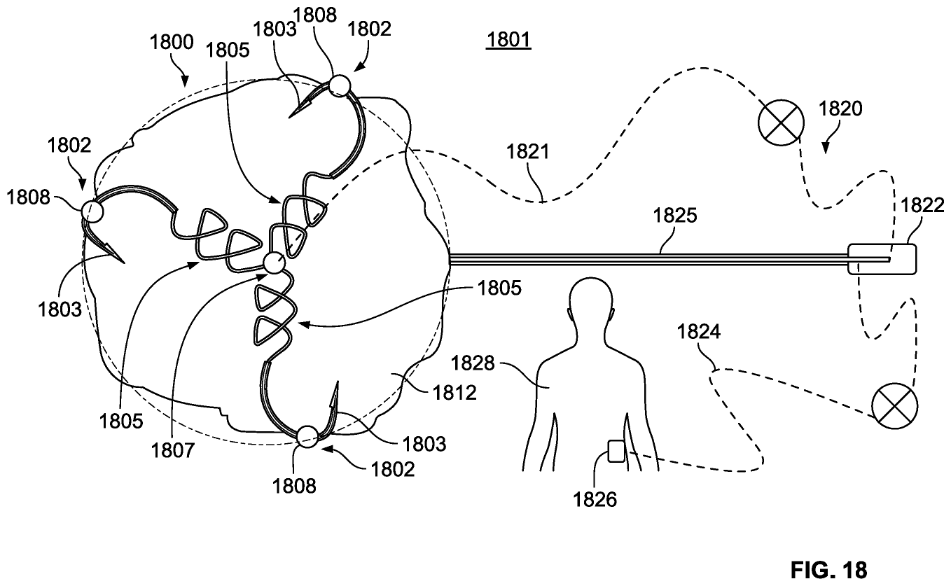

FIG. 18 shows an exemplary configuration of a system 1801 comprising an electrosurgical unit (ESU) 1820 as employed with a resection device 1800 in various embodiments of the present specification. The ESU 1820 provides a monopolar device 1822 with an internal return 1821 and an electrical path 1824 to grounding pad 1826 on the skin 1828. In other embodiments, the ESU 1820 provides a bipolar system where the electrical path passes from one electrode point in contact with one area of the targeted tissue to another electrode point in contact with another area of the targeted tissue. A resection device 1800, in FIG. 18 having members or three retraction hooks 1802, each comprising a stopper 1808 and a penetrating tip 1803, is first deployed at the target lesion 1812. The resection device 1800 further comprises a central connection hub 1807 and each member or retraction hook 1802 is connected to the central connection hub 1807 by a central portion 1805 comprising a Nitinol connector, similar to resection device 1020 shown in FIG. 10. The retraction hooks 1802 draw the edges of the lesion 1812 together, toward the center of the lesion 1812. A snare or electrosurgical knife 1825 is then used to sever the raised lesion 1812. Electrocautery is provided by the ESU 1820 to assist in removing the lesion 1812.

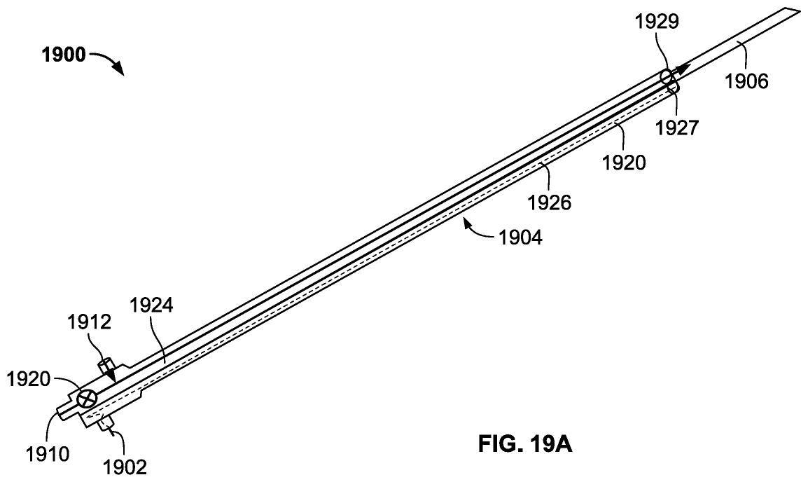

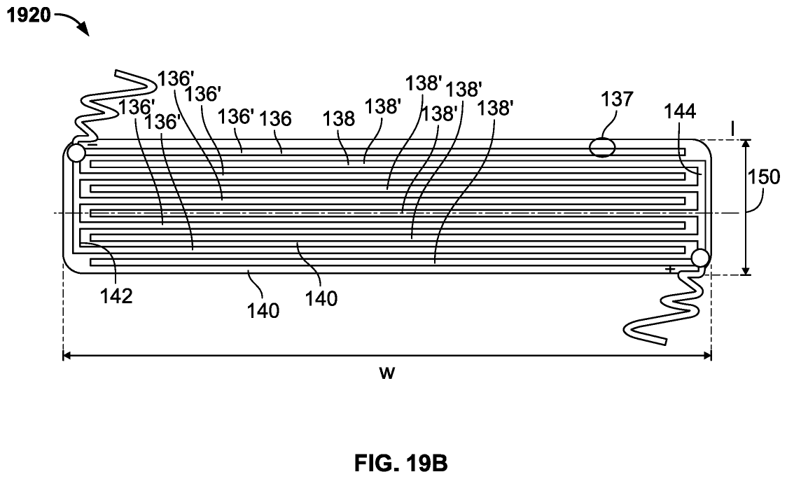

FIG. 19A shows an exemplary configuration of an electrosurgical knife or needle knife 1900 as employed with resection devices in embodiments of the present specification. In embodiments, an electrical connector 1902 is provided on an outer catheter 1904. In embodiments, the outer catheter has an outer diameter ranging from 2 to 4 mm and a length ranging from 110 to 450 cm. The electrosurgical knife or needle knife 1900 further includes a needle/knife 1906 that is coaxially deployed within the outer catheter 1904 and is extendable beyond a distal tip of the outer catheter by a distance ranging from 1 mm to 10 mm. In embodiments, the needle/knife 1906 has a gauge ranging from 28 G to 16 G. Optionally, the distal tip of the outer catheter and/or a proximal portion of needle/knife 1906 is insulated to prevent injury to nearby non-target tissues. In embodiments, electrical connector 1902 is provided to connect the needle/knife 1906 to an electrosurgical unit (ESU) 1800, shown in FIG. 18, to apply electrocautery. An input port 1910 is provided for the delivery of a fluid, such as saline. An additional side port 1912 is provided for injection of additional fluid for delivery of the fluid, via a second output port 1929 at the distal end of the outer catheter 1904, for cooling of the outer catheter 1904 or tissue area. A heating chamber 1920 is provided inline and within the outer catheter 1904 to heat the fluid delivered via the input port 1910 in a range of 50 to 100 degrees Celsius. The heating chamber 1920 is deployed anywhere along the length of the outer catheter 1904 from the catheter handle to the tip of the outer catheter 1904. In some embodiments, the heating chamber 1920 comprises a plurality of electrodes configured to receive an electrical current and heat a fluid passing through the chamber, as described below. In some embodiments, the heating chamber 1920 is configured to heat the fluid (provided from the input port 1910) for delivery via a first output port 1927 at a distal end of the outer catheter 1904 into or proximate the target tissue to assist with the resection procedure. The heating chamber 1920 is configured to heat the fluid/saline while ensuring that the fluid/saline does not vaporize. In embodiments, the outer catheter 1904 includes at least one first lumen for the delivery of a first fluid (saline) provided via the input port 1910 and heated by the heating chamber 1920, at least one second lumen 1924 for the delivery of a second fluid provided by the side port for cooling, and a wire 1926 for the delivery of electrical current to the needle/knife 1906. The at least one first lumen 1920 is in fluid communication with the input port 1910 and a first output port 1927 at the distal tip of the outer catheter 1904. The at least one second lumen 1924 is in fluid communication with the side port 1912 and the second output port 1929 at the distal tip of the outer catheter 1904. The wire 1926 is in electrical communication with the electrical connector 1902 and the needle/knife 1906. The electrical connector 1902 is connected to an ESU to provide electrocautery via the needle/knife 1906.

FIG. 19B is a longitudinal cross-sectional view 125 of a heating chamber 1920 including assembled first and second electrodes 136, 138, in accordance with some embodiments of the present specification. A plurality of electrodes, configured as first and second arrays of electrodes 136, 138, respectively comprise metal rings 142, 144 from which a plurality of electrode fins or elements 136′, 138′ extend radially and longitudinally along a longitudinal axis 150 of the heating chamber 1920. In other words, each of the electrode fins 136′, 138′ have a first dimension along a radius of the heating chamber 1920 and a second dimension along a longitudinal axis 150 of the heating chamber 1920. The electrode fins or elements 136′, 138′ define a plurality of segmental spaces 140 there-between through which saline/water flows and is heated. Electrical current is directed from a controller, into the outer catheter, through the first lumen, and to the electrodes 136, 138 which causes the fins or elements 136′, 138′ to generate heat which is then transferred to the saline in order to heat the saline. The first and second dimensions enable the electrodes 136, 138 to have increased surface area for heating the saline/water flowing in the spaces 140. In accordance with an embodiment, the first electrodes 136 have a first polarity and the second electrodes 138 have a second polarity opposite said first polarity. In an embodiment, the first polarity is negative (cathode) while the second polarity is positive (anode). The electrodes 136, 138 (including rings 142, 144 and fins or elements 136′, 138′) are all flexible to allow for bending of the distal portion or tip of the catheter to provide better positioning of the outer catheter during resection procedures.

As shown in FIG. 19B, when the heating chamber 1920 is assembled, the electrode fins or elements 136′, 138′ interdigitate or interlock with each other (similar to fingers of two clasped hands) such that a cathode element is followed by an anode element which in turn is followed by a cathode element that is again followed by an anode element and so on, with a space 140 separating each cathode and anode element. In various embodiments, each space 140 has a distance from a cathode element to an anode element ranging from 0.01 mm to 2 mm. In some embodiments, the first array of electrodes 136 has a range of 1 to 50 electrode fins 136′, with a preferred number of 4 electrode fins 136′, while the second array of electrodes 138 has a range of 1 to 50 electrode fins 138′, with a preferred number of 4 electrode fins 138′. In various embodiments, the heating chamber 130 has a width w in a range of 1 to 5 mm and a length 1 in a range of 5 to 50 mm. In some embodiments, the heating chamber 1920 includes at least one sensor 137. In various embodiments, said at least one sensor 137 comprises an impedance, temperature, pressure or flow sensor, with the pressure sensor being less preferred. In one embodiment, the electrical impedance of the electrode arrays 136, 138 can be sensed. In other embodiments, the temperature of the fluid, temperature of the electrode arrays, fluid flow rate, pressure, or similar parameters can be sensed.

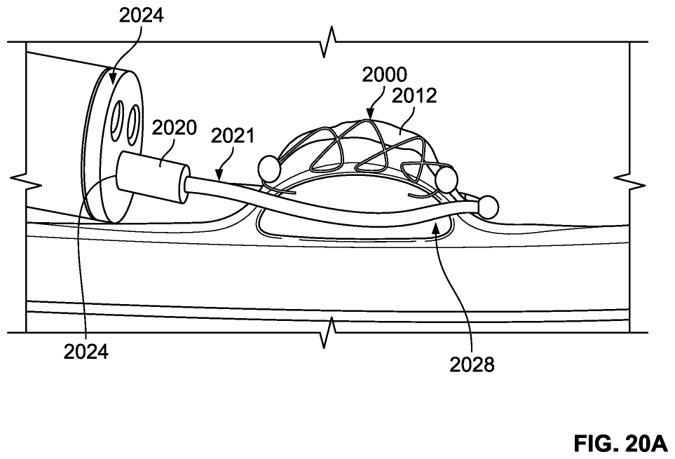

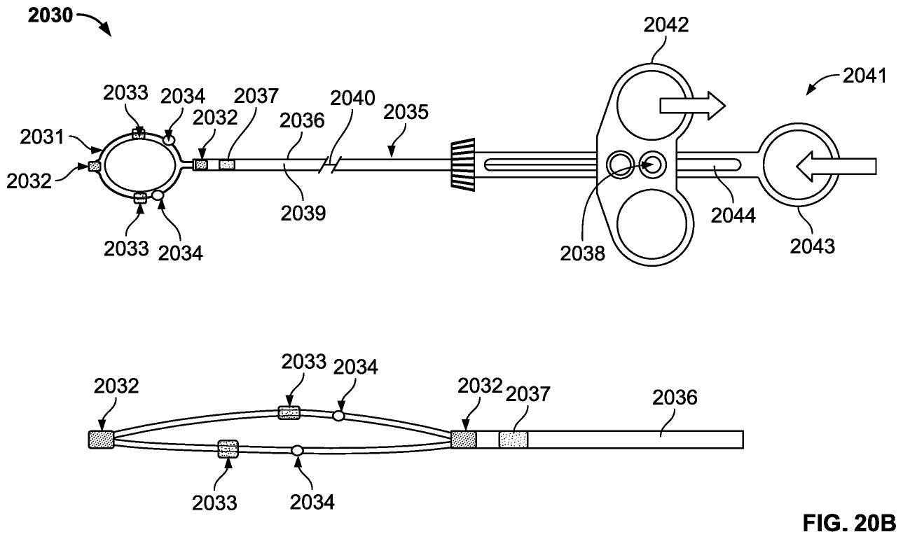

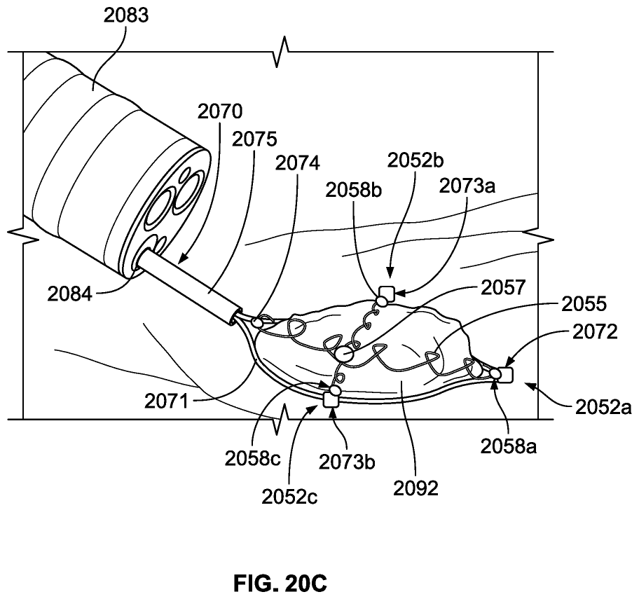

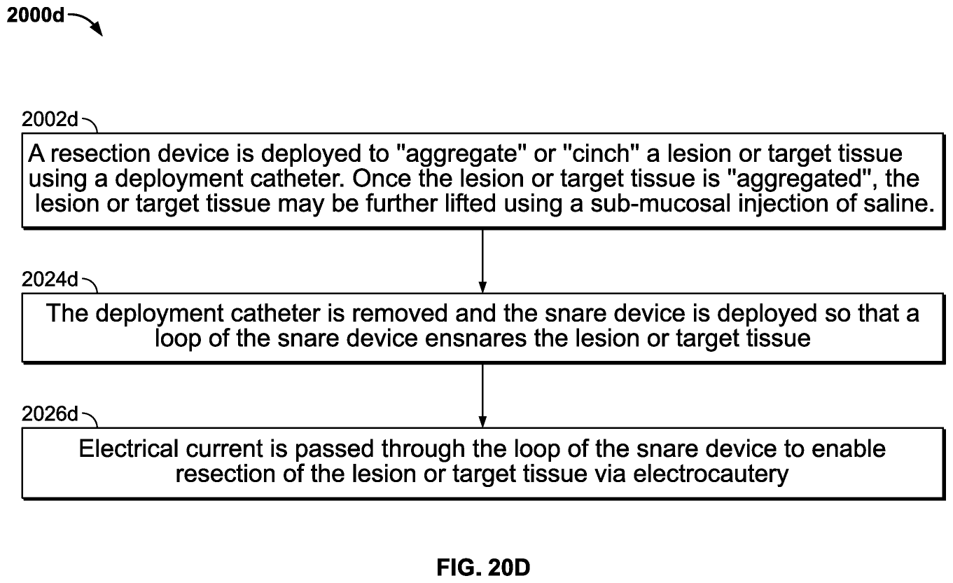

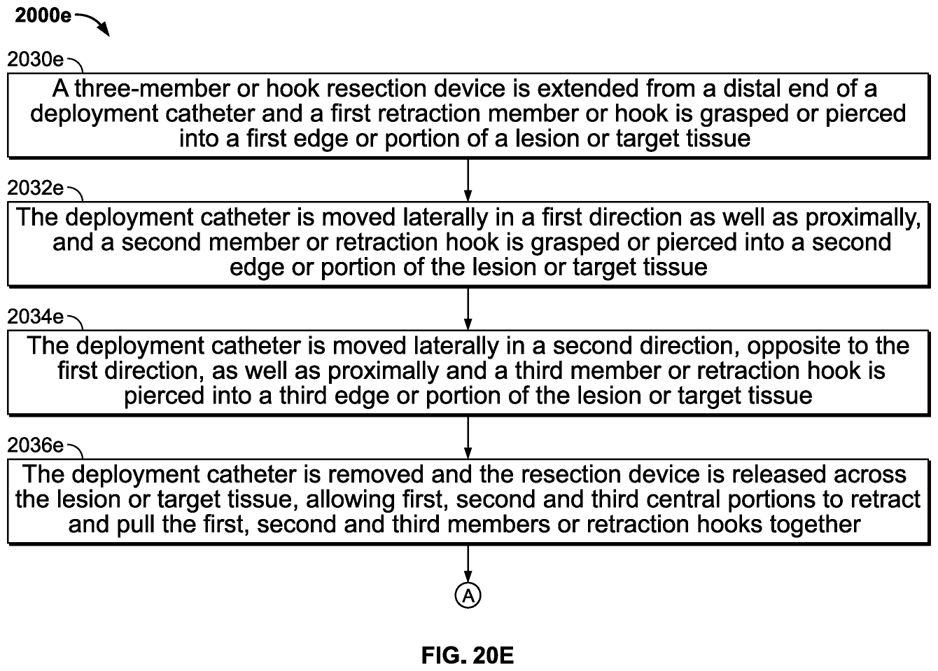

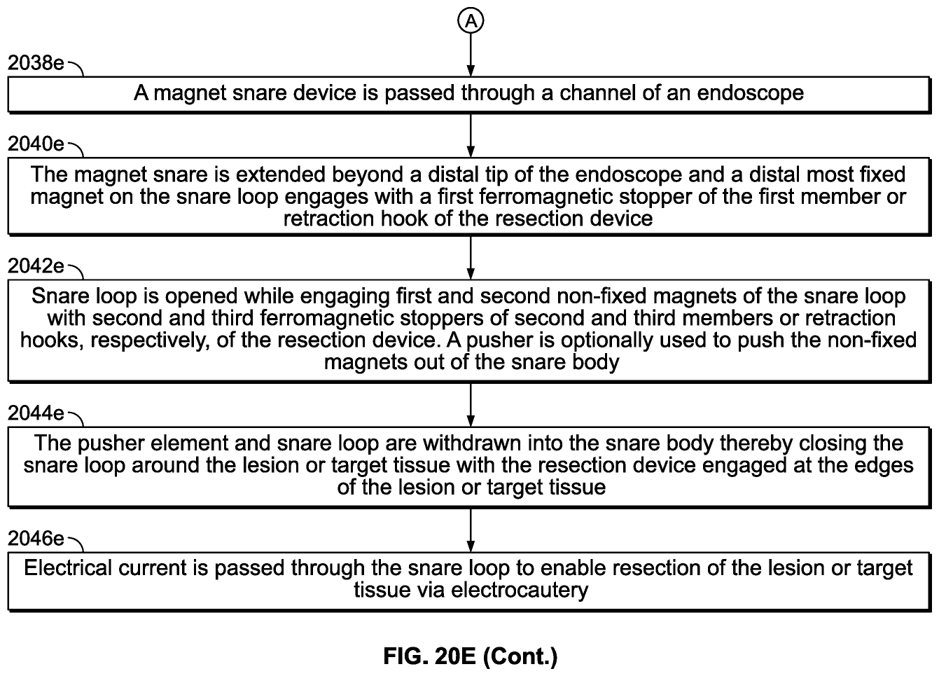

FIG. 20A is an illustration of a snare device as used with a resection device in a resection procedure in embodiments of the present specification. A snare device 2020 is passed through a channel 2023 of an endoscope 2024. A resection device 2000 is deployed to “aggregate” or “cinch” a lesion 2012. Once the lesion 2012 is “aggregated”, the lesion 2012 may be further lifted using a sub-mucosal injection 2028 of saline or another lifting agent known to persons of ordinary skill in the art. Subsequently, the snare device 2020 is used to resect the lesion 2012. Electrical current is provided to the loop 2021 of the snare device which is used to remove the lesion 2012 via electrocautery. In some embodiments, the snare device used is similar to the snare device disclosed in United States Patent Application Publication Number US 2017-0007279 A1, which is herein incorporated by reference in its entirety.

In some embodiments, the snare device further includes magnetic or ferromagnetic elements to connect with one or more of the stoppers on the retraction hooks to secure a portion of the snare loop of the snare device to the retraction hooks of the resection device. In various embodiments, the magnetic or ferromagnetic elements are either fixed to the wire of the snare loop of the snare resection device or are freely mobile along the wire of the snare loop. In various embodiments, the polarity of these magnetic or ferromagnetic elements are arranged such that they repel the adjacent magnetic/ferromagnetic element so as to not clump to each other while on the snare, thereby preventing the interruption of the opening and closing function of the snare device. In various embodiments, the magnetic/ferromagnetic elements are designed to engage with magnetic or ferromagnetic elements of the retraction hook.

FIG. 20B is an embodiment of a magnet snare device 2030 configured to operate in conjunction with a resection device of the present specification. The snare loop 2031 is composed of a non-ferromagnetic material such as nylon or Nitinol. At least one first magnet 2032 is positioned coaxially on or attached to the wire of the snare loop 2031 and is relatively immobile relative to the length of the snare loop 2031. At least one second magnet 2033 is threaded on the snare loop 2031 and is relatively mobile relative to the length of the snare loop 2031. The magnet snare device 2030 further includes at least one fixed non-ferromagnetic pusher element 2034 positioned coaxially on or attached to the snare loop 2031 and positioned proximal to each of the mobile magnets 2033 that is configured to push the mobile magnets 2033 out of the body 2035 of the magnet snare device 2030. In embodiments, the pusher element 2034 is a bead fixed to the snare loop 2031 configured to push the mobile magnet 2033 out of the body 2035 and prevent proximal movement of the mobile magnet 2033 along the snare loop 2031. In some embodiments, at least one third magnet 2037 is attached to a snare sheath 2036. In various embodiments, the at least one third magnet is fixed to or detachable from the snare sheath 2036.

The polarity of the mobile magnets 2033 is arranged such that the two mobile magnets 2033 on the snare loop 2031 repel each other. The magnet snare device 2030 includes an electrical surgical connector 2038 for connection to an ESU. The magnet snare device 2030 is configured to pass electrosurgical current from the electrical surgical connector 2038 through a wire 2040 extending through the magnetic snare device 2030 to the snare loop 2031 or alternate energy through a lumen 2039 of the snare sheath 2036. The magnet snare device 2030 further includes a handle 2041 for actuating the device 2030. In embodiments, the handle 2041 includes a first finger hole portion 2042 positioned coaxially about, and longitudinally slidable within a slot 2044 of, a second finger hole portion 2043. In embodiments, sliding the first finger hole portion 2042 in a proximal direction while moving the second finger hole portion 2043 in a distal direction closes the snare loop 2031.

FIG. 20C illustrates a resection of a lesion using a magnet snare device 2070 in conjunction with a resection device 2050, in accordance with embodiments of the present specification. The snare device 2070 is passed through a channel 2083 of an endoscope 2084. The resection device 2050 is deployed to “aggregate” or “cinch” a lesion 2092. A distal most fixed magnet 2072 on the snare loop 2071 engages with a first ferromagnetic stopper 2058a of a first member or retraction hook 2052a of the resection device 2050. The member or retraction hook 2052a is connected to a central connection hub 2057 by a central portion 2054 or coil of the resection device 2050.

As the snare loop 2071 is opened, two non-fixed magnets 2073a, 2073b engage with the one or more ferromagnetic stoppers 2058b, 2058c on second and third members or retraction hooks 2052b, 2052c of the resection device 2050. An optional pusher element 2074 is used to push the magnets 2073a, 2073b out of the snare body 2075 and maintain the position of these magnets 2073a, 2073b in the distal 80% of the snare loop 2071. While closing the snare loop 2071, the pusher element 2074 is withdrawn into the snare body 2075 and does not materially interfere with the functioning of the snare device 2070. The snare loop 2071 wire slides relative to the mobile magnets 2073a, 2073b, further cinching the lesion 2092 while maintaining the position of the wire relative to the stoppers and lesion, prior to and during electrocautery or an alternate respective energy application. The fixed magnet 2072 coupled to the distal ferromagnetic stopper 2058a pulls the distal edge of the lesion 2092 toward the snare body 2075, further cinching the lesion 2092 prior to and during electrocautery or an alternate respective energy application.

FIG. 20D is a flowchart of a plurality of exemplary steps of a method 2000d of using the snare device of FIG. 20A with a resection device in a resection procedure, in accordance with some embodiments of the present specification. In various embodiments, the resection device could be one with two members or retraction hooks (as illustrated with reference to FIG. 1) or one with three members or retraction hooks (as illustrated with reference to FIGS. 10A and 10B).

At step 2002d, the resection device is deployed to “aggregate” or “cinch” a lesion or target tissue. In some embodiments, once the lesion or target tissue is “aggregated”, the lesion or target tissue may be further lifted using a sub-mucosal injection of saline or another lifting agent known to persons of ordinary skill in the art. In some embodiments, the resection device is deployed by extending from a distal end of a deployment catheter and maneuvered to engage two or more retraction hooks of the resection device with two or more edges or regions of the lesion or target tissue. In some embodiments, the deployment catheter is passed through a channel of an endoscope.

At step 2004d, the deployment catheter is removed and the snare device is deployed so that a loop of the snare device ensnares the lesion or target tissue. In some embodiments, the snare device is deployment by passing the snare device through the channel of the endoscope. At step 2006d, electrical current is passed through the loop of the snare device to enable resection of the lesion or target tissue via electrocautery.

FIG. 20E is a flowchart of a plurality of exemplary steps of a method 2000e of using the magnet snare device of FIG. 20B with a three-member or hook resection device in a resection procedure, in accordance with some embodiments of the present specification. At step 2030e, a three-member hook resection device is extended from a distal end of a deployment catheter and a first member or retraction hook is pierced into a first edge or portion of a lesion or target tissue. At step 2032e, the deployment catheter is moved laterally in a first direction as well as proximally, and a second member or retraction hook is pierced into a second edge or portion of the lesion or target tissue. At step 2034e, the deployment catheter is moved laterally in a second direction, opposite to the first direction, as well as proximally and a member or third retraction hook is pierced into a third edge or portion of the lesion or target tissue. At step 2036e, the deployment catheter is removed and the resection device is released across the lesion or target tissue, allowing first, second and third central portions (each of which connects the first, second and third retraction hooks to a central connection hub or are attached together) to retract and pull the first, second and third retraction members or hooks together such that the first, second and third edges or portions of the lesion or target tissue are closer together and the edges or portions of the lesion or target tissue are pulled approximately toward a center of the lesion or target tissue. At step 2038e, a magnet snare device is passed through a channel of an endoscope. At step 2040e, the magnet snare is extended beyond a distal tip of the endoscope and a distal most fixed magnet on the snare loop engages with a first ferromagnetic stopper of the first member or retraction hook.

At step 2042e, the magnet snare loop is opened while engaging first and second non-fixed magnets of the snare loop with second and third ferromagnetic stoppers of second and third members or retraction hooks, respectively, of the resection device. In some embodiments, an optional pusher element is used to push the first and second non-fixed magnets out of the snare body and maintain the position of the first and second non-fixed magnets in a distal 80% of the snare loop.

At step 2044e, the pusher element and snare loop are withdrawn into the snare body, thereby closing the snare loop around the lesion or target tissue with the resection device engaged at the edges of the lesion or target tissue. The snare loop wire slides relative to the first and second non-fixed/mobile magnets further cinching the lesion or target tissue. At step 2046e, electrical current is passed through the loop of the snare device to enable resection of the lesion or target tissue via electrocautery.

FIG. 20F illustrates a variable size snare device 2100f for use in conjunction with the resection devices of the present specification. The snare device 2100f comprises a handle 705 coupled to a snare sheath 715. The snare sheath 715 has a proximal end 716 which is coupled to the handle 705 and a distal end 717 which is coupled to a snare loop 710. A wire, such as a pair of wires 720 shown in the embodiment of FIG. 2100f, is disposed within the snare sheath 715 such that a proximal end of the wire 720 extends from the proximal end 716 of the sheath 715 and is connected to the handle 705 and a distal end of the snare wire 720 extends from the distal end 717 of the sheath 715 and is connected to the snare loop 710. The snare sheath 715 and the wire 720 present in the snare sheath 715 are controlled by an operator through the handle 705.

In an embodiment, the snare loop 710 comprises a first section 761 and a second section 762 wherein the distal ends of both first section 761 and the second section 762 are coupled to a third section 763. The proximal ends of first section 761 and second section 762 are coupled to the snare wire 720 that is disposed within the snare sheath 715. In an embodiment, the third section 763 comprises almost half of the total perimeter of the snare loop 710 and is substantially circular in shape. In an embodiment, the first section 761 further comprises a plurality of sub sections such as sub sections 761a, 761b and 761c and the second section 762 comprises a plurality of subsections such as sub sections 762a, 762b and 762c. In embodiments, each of subsections such as 761a, 761b, 761c, 762a, 762b and 76c is coupled to other sections of the snare loop at different angulations 711. In an embodiment, the length and angular orientation of subsection 761a is aligned to that of subsection 762a and the length and angular orientation of subsection 761b is aligned to that of subsection 762b and the length and angular orientation of subsection 761c is aligned to that of subsection 762c. In embodiments, the size of the snare loop 710 can be modified by extending or retreating portions of first section 761 and second section 762 in the sheath 715. In an embodiment, the size of the snare loop 710 can be modified in discrete steps to reduce the size of the snare loop 710 such that subsections 761a and 762a are simultaneously withdrawn inside the snare sheath 715. Similarly, subsections 761b and 762b can be simultaneously withdrawn inside the snare sheath 715 to further reduce the size of the snare loop 710. To even further reduce the size of the snare loop 710, subsections 761c and 762c are also withdrawn into the snare sheath 715. As the corresponding subsections, such as subsections 761a and 762a, are aligned with each other in terms of length and angular orientation, as the size of the snare loop 710 is modified in discrete steps, the snare loop 710 substantially maintains its original shape. In other words, the subsections 761a, 761b, 761c, 762a, 762b and 762c, due to their length and angular orientations, ratchet down the third section 763, as the size of the snare loop 710 is reduced in discrete steps, while still substantially maintaining the original shape of the loop 710.

In an embodiment, the snare loop 710 is manufactured from shape memory alloy such as Nitinol and has two different stiffness levels across certain ranges of temperature. In an embodiment, snare loop 710 has a first stiffness level below 30 degrees Celsius temperature and a second stiffness level above 30 degrees Celsius temperature, wherein the second stiffness is greater than the first stiffness.

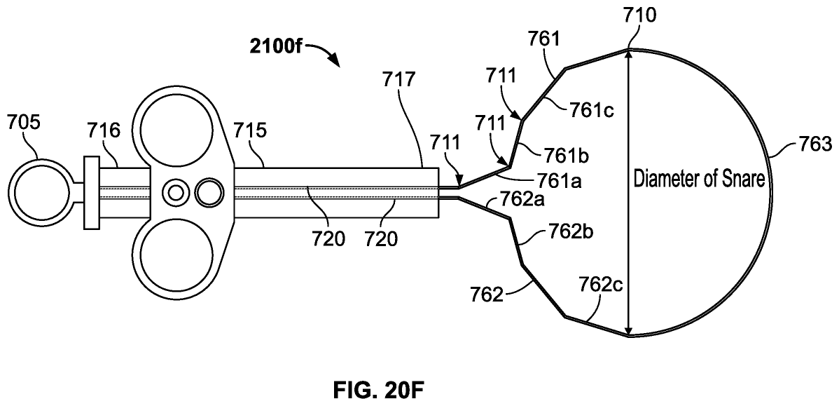

FIG. 21 is an illustration of a submucosal dissection procedure using a resection device 2100 in accordance with embodiments of the present specification. In embodiments, the resection device 2100 has been deployed on a lesion 2112. As an edge 2113 of the lesion 2112 is dissected, the central portion 2104 of the resection device 2100 coils further (as dissected edge 2113 of the lesion is free and can be pulled back by member or retraction hook 2102), exposing the base 2114 of the lesion. The coiling mechanism of the resection device 2104 lifts and retracts the dissecting edge 2113 of the lesion 2112 towards a center of the lesion 2112 and away from the point of dissection as the edge 2113 is resected (in some embodiments, using knife or scissor 2120 via endoscope 2122), assisting in the resection.

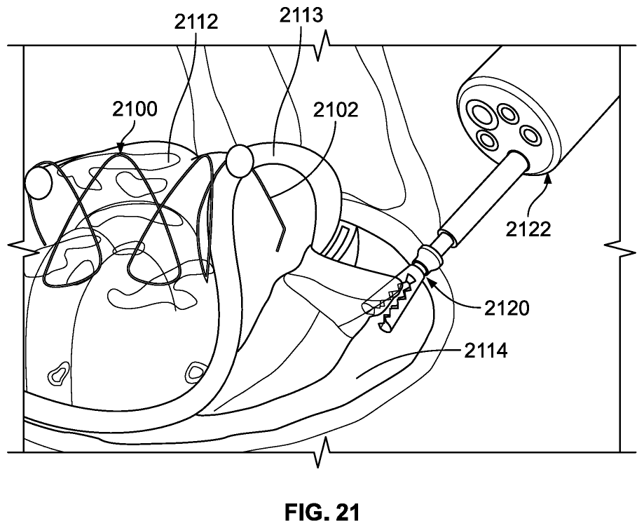

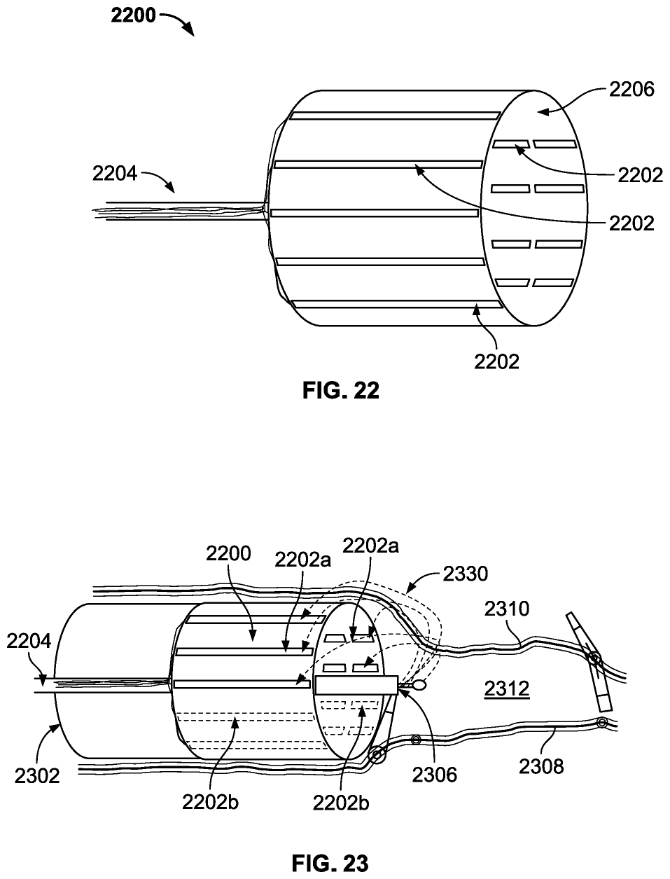

FIG. 22 is a diagram of a directional grounding cap 2200 in accordance with embodiments of the present specification. FIG. 23 is a diagram showing the function of the directional grounding cap shown in FIG. 22 in accordance with embodiments of the present specification. Referring to both FIGS. 22 and 23, in embodiments, a plurality of grounding electrodes 2202 are distributed around the circumference of a directional grounding cap 2200. The directional grounding cap 2200 further comprises a grounding cable 2204 and a distal attachment cap 2206. In embodiments, the directional grounding cap 2200 is electrically connected to an endoscope 2302 via a grounding cable 2204 and an electrosurgical catheter 2306. Grounding electrodes 2202 can be selected individually or in groups to direct the flow 2330 of electrosurgical current away from selected healthy tissue towards the selected diseased tissue, preventing electrosurgical damage to the selected healthy tissue. In embodiments, a first set of grounding electrodes 2202a are turned ‘on’ and a second set of grounding electrodes 2202b are turned ‘off’ the electrical energy is directed away from the muscularis propria 2308, toward a mucosa 2310 or a submucosal space 2312. An ESU can sample the impedance values for each of the grounding electrode 2202 and automatically select the best grounding electrode to perform electrocautery or electrosurgical.

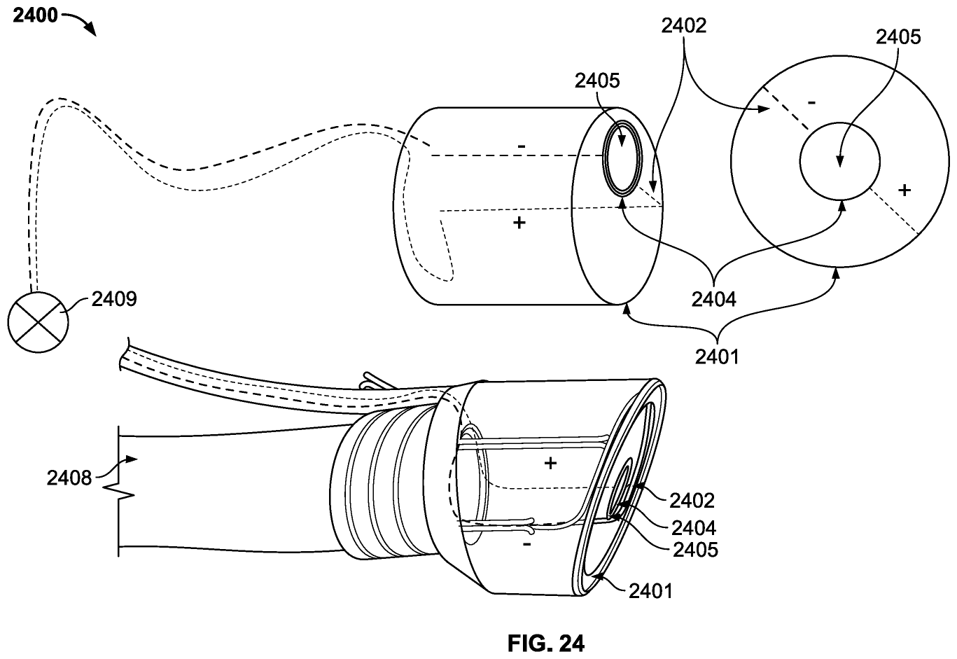

FIG. 24 is a diagram of an embodiment of a bipolar cap dissector 2400 in accordance with embodiments of the present specification. The bipolar cap dissector 2400 includes a cap or housing 2401 that has at least one cutting wire 2402 connected with an insulated ring/bead/sphere structure 2404. The bipolar cap dissector 2400 includes a central opening 2405 for engaging with a catheter passed through a biopsy channel of an endoscope 2408 to manipulate the ring 2404 to improve the contact of the at least one cutting wire 2402 with tissue. The catheter has a steerable tip which can be steered in one or more directions to move the ring/bead/sphere 2404 and attached at least one cutting wire 2402 to improve contact with the tissue to be dissected. In embodiments, the at least one cutting wire 2402 includes a mechanism for creating slack in the wire to be able to push the wire 2402 and the ring 2404 out of its position from within the cap. In embodiments, the cutting wire 2402 has a mechanism for creating slack wherein the ring/bead/sphere 2404 can be extended by a distance ranging from 1 mm to 50 mm. In an embodiment, the cutting wire 2402 serves as a first electrode (either anode or cathode) while an inserted catheter serves as the second electrode (opposite polarity to the first electrode). In embodiments, the cutting wire 2402 may be partially insulated along its length from the cap to the insulated ring 2404. In embodiments, the insulated ring 2404 is offset from the center of the cap 2401 and aligned with the channel of the endoscope 2408 to be able to engage with the channel of the endoscope 2408. In one embodiment, the insulated ring 2404 is offset away from the camera of the endoscope 2408 so as not to interfere with the visualization using the endoscope 2408. In embodiments, the internal diameter of the insulated ring/bead/sphere 2404 ranges from greater than or equal to 0.1 mm to less than or equal to 10 mm. In one embodiment, both the electrical electrodes are connected to an ESU 2409 using the catheter.

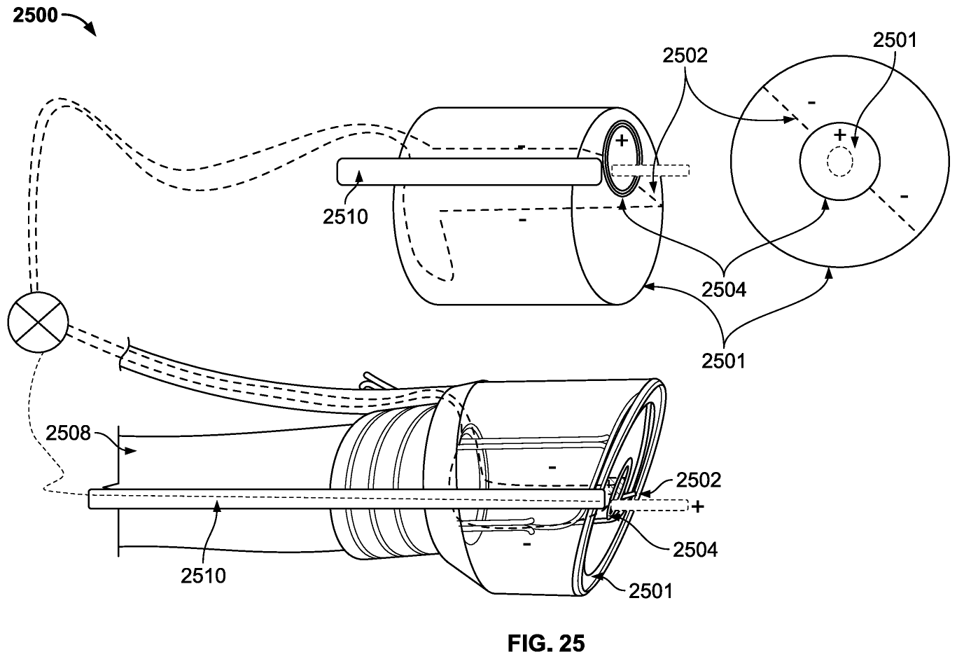

FIG. 25 is a diagram of another embodiment of a bipolar cap dissector for use with embodiments of the present specification. The bipolar cap dissector 2500 includes at least one cutting wire 2502 connected to an insulated ring/bead/sphere structure 2504 that includes a central opening 2501 for engaging with an electrosurgical needle or knife 2510. The electrosurgical needle or knife 2510 is passed through the channel of an endoscope 2508 to manipulate the ring 2504 in order to improve the contact of the at least one cutting wire 2402 with tissue. In an embodiment, the at least one cutting wire 2502 serves as a first electrode while the electrosurgical needle/knife 2510 serves as the second electrode. In embodiments, the at least one cutting wire 2502 includes a mechanism for creating slack to be able to push the wire 2502 and the ring 2504 out of its position from within the cap or housing 2501. In embodiments, the at least one cutting wire 2502 has a mechanism for creating slack wherein the ring/bead/sphere 2504 can be extended by a distance ranging from 1 mm to 50 mm. In embodiments, the at least one cutting wire 2502 may be partially insulated along its length from the cap to the insulated ring 2504. In embodiments, the insulated ring 2504 is offset from the center and aligned with the channel of the endoscope 2508 to be able to engage with the channel of the endoscope 2508. In embodiments, the internal diameter of the insulated ring/bead/sphere 2504 ranges from greater than or equal to 0.1 mm to less than or equal to 5 mm.

In various embodiments saline heated between 50° C. and 100° C. is injected to create coagulation of small blood vessel in order to prevent bleeding during electrosurgery while at the same time providing a lifting function separating various tissue layers to create a dissection plane. Alternate sources of energy such as microwave, laser, cryogen can be used to heat the fluid or the tissue. The amount of energy delivered ranges from 50 J to 50,000 J in one treatment session and 1 J to 50 J per discrete energy application.

In various embodiments, the ESU blends an electrosurgical RF (Radio Frequency) current of >1 KHz with an electrostimulation RF current of <1 KHz to prevent or treat hemorrhage during electrosurgery. The frequency of the electrostimulation RF current can be in the range of approximately 1 microhertz (μHz) to about 1 kilohertz (KHz), although the methods described herein may be practiced by administering electrostimulation RF current having a frequency outside of this range. Typically, an electrostimulation RF current may have a frequency of about 1 mHz to about 1 KHz such as, for example, a frequency of from about 0.1 Hz to about 10 Hz. In certain embodiments, an electrical stimulus may be administered at a frequency of 1 Hz. In certain embodiments, an electrical stimulus may be administered at a frequency of 10 Hz. Also, certain treatments may include multiple electrical stimuli including any combination of frequencies. Specific ranges of electrostimulation RF current which may be used in the present specification to prevent or treat hemorrhage during an electrosurgical resection procedure are also disclosed in U.S. Pat. No. 10,603,489, which is herein incorporated by reference in its entirety.

In various embodiments, other fixing methods known in the art such as articulating clips or magnets can be used in place of the hook element to fixate the device into the tissue.

The above examples are merely illustrative of the many applications of the systems and methods of present specification. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

We claim:

1. A device for performing an endoscopic resection comprising:
a first member, wherein the first member comprises at least one first penetrating tip for piercing a first position on a lesion inside a patient;
a second member, wherein the second member comprises at least one second penetrating tip for piercing a second position on the lesion inside the patient; and
a central portion having a first end and a second end, wherein the first member is attached to the first end of the central portion and the second member is attached to the second of the central portion, wherein the central portion is configured to change shape from a first linear configuration to a second coiled configuration, wherein, upon attachment of the device to the lesion, the at least one first member is configured to grasp the first position, the at least one second member is configured to grasp the second position, and the central portion is configured to change shape from the first linear configuration to the second coiled configuration to contract and thereby compress the lesion and reduce at least one dimension of the lesion.

2. The device of claim 1, further comprising a deployment catheter configured to contain the device and maintain the central portion in the first linear configuration.

3. The device of claim 2, wherein the central portion is configured to change shape to the second coiled configuration upon exiting the deployment catheter.

4. The device of claim 1, wherein the central portion comprises at least one of a spring, coil, or elastic band.

5. The device of claim 1, wherein the central portion comprises a shape memory material.

6. The device of claim 5, wherein the shape memory material is Nitinol.

7. The device of claim 1, wherein the first position is positioned opposite the second position around an edge of the lesion.

8. The device of claim 1, further comprising a third member, wherein the third member comprises at least one third penetrating tip for piercing a third position on the lesion inside the patient.

9. The device of claim 8, wherein the first position, second position, and third position are equidistantly spaced around an edge of the lesion.

10. A method of performing an endoscopic resection comprising:
providing a resection device comprising:
a first member, wherein the first member comprises at least one first penetrating tip for piercing a first position on a lesion inside a patient;
a second member, wherein the second member comprises at least one second penetrating tip for piercing a second position on the lesion inside the patient;
a central portion having a first end and a second end, wherein the first member is attached to the first end of the central portion and the second member is attached to the second of the central portion, wherein the central portion is configured to change shape from a first linear configuration to a second coiled configuration; and
a deployment catheter configured to contain the device and maintain the central portion in the first linear configuration;
extending the resection device from a distal end of the deployment catheter such that the at least one first penetrating tip pierces into the first position;
retracting the catheter such that the resection device is pulled across the lesion and the at least one second penetrating tip of the second member pierces into the second position; and
removing the catheter and releasing the central portion across the lesion, allowing the central portion to change shape from the first linear configuration to the second coiled configuration to thereby compress the lesion and reduce at least one dimension of the lesion.

11. The method of claim 10, further comprising lifting the lesion using a sub-mucosal injection to lift the lesion off a muscularis of the patient.

12. The method of claim 11, further comprising using an electrosurgical device to remove the lesion.

13. The method of claim 12, wherein the electrosurgical device is a needle/knife.

14. The method of claim 10, wherein the deployment catheter further comprises a lumen through which fluid may be passed and injected into or proximate the lesion.

15. The method of claim 14, wherein the fluid is heated saline and wherein a temperature of the heated saline is between 50° C. and 100° C.

16. The method of claim 10, further comprising at least one stopper positioned proximate the first member or the second member, wherein at least one dimension of the at least one stopper is greater than a diameter of the first member or the second member.

17. The method of claim 10, wherein the central portion comprises at least one of a spring, coil, elastic band, or a shape memory material.

18. The method of claim 10, wherein the shape memory material is Nitinol.
